SEQUENCE LISTING (44 sequences)

SEQ ID NO: 1 - DNA, 18 bp, Artificial Sequence
  Description of Artificial Sequence Synthetic polynucleotide sequence
tttttttttttttttttt

SEQ ID NO: 2 - DNA, 9 bp, Artificial Sequence
  Description of Artificial Sequence Synthetic polynucleotide sequence
ttttttttt

SEQ ID NO: 3 - DNA, 18 bp, Artificial Sequence
  Description of Artificial Sequence Synthetic polynucleotide sequence
ttatattttttctttccc

SEQ ID NO: 4 - DNA, 9 bp, Artificial Sequence
  Description of Artificial Sequence Synthetic polynucleotide sequence
ttatatttt

SEQ ID NO: 5 - DNA, 18 bp, Artificial Sequence
  Description of Artificial Sequence Synthetic polynucleotide sequence
ttatattttctctttccc

SEQ ID NO: 6 - DNA, 9 bp, Artificial Sequence
  Description of Artificial Sequence Synthetic polynucleotide sequence
ttatatttt

SEQ ID NO: 7 - DNA, 18 bp, Artificial Sequence
  Description of Artificial Sequence Synthetic polynucleotide sequence
tttttttttttttttttt

SEQ ID NO: 8 - DNA, 4 bp, Artificial Sequence
  Description of Artificial Sequence Synthetic polynucleotide sequence
tttt

SEQ ID NO: 9 - DNA, 9 bp, Artificial Sequence
  Description of Artificial Sequence Synthetic polynucleotide sequence
ttttttttt

SEQ ID NO: 10 - DNA, 12 bp, Artificial Sequence
  Description of Artificial Sequence Synthetic polynucleotide sequence
tttttttttttt

SEQ ID NO: 11 - DNA, 18 bp, Artificial Sequence
  Description of Artificial Sequence Synthetic polynucleotide sequence
tttttttttutttttttt

SEQ ID NO: 12 - DNA, 18 bp, Artificial Sequence
  Description of Artificial Sequence Synthetic polynucleotide sequence
ttttttttuutttttttt

SEQ ID NO: 13 - DNA, 18 bp, Artificial Sequence
  Description of Artificial Sequence Synthetic polynucleotide sequence
ttatattttttctttccc

SEQ ID NO: 14 - DNA, 9 bp, Artificial Sequence
  Description of Artificial Sequence Synthetic polynucleotide sequence
ttatatttt

SEQ ID NO: 15 - DNA, 18 bp, Artificial Sequence
  Description of Artificial Sequence Synthetic polynucleotide sequence
ttatattttctctttccc

SEQ ID NO: 16 - DNA, 18 bp, Artificial Sequence
  Description of Artificial Sequence Synthetic polynucleotide sequence
ttatattttttctttccc

SEQ ID NO: 17 - DNA, 9 bp, Artificial Sequence
  Description of Artificial Sequence Synthetic polynucleotide sequence
ttatatttt

SEQ ID NO: 18 - DNA, 18 bp, Artificial Sequence
  Description of Artificial Sequence Synthetic polynucleotide sequence
attccgtcatcgctcctc

SEQ ID NO: 19 - DNA, 9 bp, Artificial Sequence
  Description of Artificial Sequence Synthetic polynucleotide sequence
attccgtca

SEQ ID NO: 20 - DNA, 18 bp, Artificial Sequence
  Description of Artificial Sequence Synthetic polynucleotide sequence
attccgtcaccgctcctc

SEQ ID NO: 21 - DNA, 18 bp, Artificial Sequence
  Description of Artificial Sequence Synthetic polynucleotide sequence
attccgtcatcgctcctc

SEQ ID NO: 22 - DNA, 9 bp, Artificial Sequence
  Description of Artificial Sequence Synthetic polynucleotide sequence
attccgtca

SEQ ID NO: 23 - DNA, 20 bp, Artificial Sequence
  Description of Artificial Sequence Synthetic polynucleotide sequence
tattccgtcatcgctcctca

SEQ ID NO: 24 - DNA, 12 bp, Artificial Sequence
  Description of Artificial Sequence Synthetic polynucleotide sequence
tattccgtcatc

SEQ ID NO: 25 - DNA, 20 bp, Artificial Sequence
  Description of Artificial Sequence Synthetic polynucleotide sequence
tattccgtcatcgctcctca

SEQ ID NO: 26 - DNA, 12 bp, Artificial Sequence
  Description of Artificial Sequence Synthetic polynucleotide sequence
tattccgtcatc

SEQ ID NO: 27 - RNA, 18 nt, Artificial Sequence
  Description of Artificial Sequence Synthetic polynucleotide sequence
aaaaaaaaaaaaaaaaaa

SEQ ID NO: 28 - RNA, 18 nt, Artificial Sequence
  Description of Artificial Sequence Synthetic polynucleotide sequence
gggaaagaaaaaauauaa

SEQ ID NO: 29 - RNA, 40 nt, Artificial Sequence
  Description of Artificial Sequence Synthetic polynucleotide sequence
cgcaggccccugaggagcgaugacggaauauaagcuggug

SEQ ID NO: 30 - DNA, 36 bp, Artificial Sequence
  Description of Artificial Sequence Synthetic polynucleotide sequence
agctatctcgagatgagctggcttctgttcctggcc

SEQ ID NO: 31 - DNA, 36 bp, Artificial Sequence
  Description of Artificial Sequence Synthetic polynucleotide sequence
ggccgcaagctttcagtcttccgattgtttagctcc

SEQ ID NO: 32 - DNA, 8 bp, Artificial Sequence
  Description of Artificial Sequence Synthetic polynucleotide sequence
tttttttt

SEQ ID NO: 33 - DNA, 8 bp, Artificial Sequence
  Description of Artificial Sequence Synthetic polynucleotide sequence
tctttccc

SEQ ID NO: 34 - DNA, 9 bp, Artificial Sequence
  Description of Artificial Sequence Synthetic polynucleotide sequence
ttctttccc

SEQ ID NO: 35 - DNA, 13 bp, Artificial Sequence
  Description of Artificial Sequence Synthetic polynucleotide sequence
ttttttttttttt

SEQ ID NO: 36 - DNA, 8 bp, Artificial Sequence
  Description of Artificial Sequence Synthetic polynucleotide sequence
tttttttt

SEQ ID NO: 37 - DNA, 5 bp, Artificial Sequence
  Description of Artificial Sequence Synthetic polynucleotide sequence
ttttt

SEQ ID NO: 38 - DNA, 8 bp, Artificial Sequence
  Description of Artificial Sequence Synthetic polynucleotide sequence
tctttccc

SEQ ID NO: 39 - DNA, 9 bp, Artificial Sequence
  Description of Artificial Sequence Synthetic polynucleotide sequence
ttctttccc

SEQ ID NO: 40 - DNA, 8 bp, Artificial Sequence
  Description of Artificial Sequence Synthetic polynucleotide sequence
cgctcctc

SEQ ID NO: 41 - DNA, 8 bp, Artificial Sequence
  Description of Artificial Sequence Synthetic polynucleotide sequence
cgctcctc

SEQ ID NO: 42 - DNA, 7 bp, Artificial Sequence
  Description of Artificial Sequence Synthetic polynucleotide sequence
ctcctca

SEQ ID NO: 43 - DNA, 7 bp, Artificial Sequence
  Description of Artificial Sequence Synthetic polynucleotide sequence
ctcctca

SEQ ID NO: 44 - DNA, 18 bp, Artificial Sequence
  Description of Artificial Sequence Synthetic polynucleotide sequence
gggaaagaaaaaatataa

(19) United States
(12) Patent Application Publication
(10) Pub. No.: US 2005/0233455 A1
(43) Pub. Date: Oct. 20, 2005

(54) Acyclic linker-containing oligonucleotides and uses thereof

(76) Inventors: Masad J. Damha, St.Hubert (CA); Ekaterina Viazovkina, Bothell, WA (US); Maria M. Mangos, Montreal (CA); Michael A. Parniak, Pittsburg, PA (US); Kyung-Lyum Min, Montreal (CA)

Correspondence Address: DAVID S. RESNICK, 100 SUMMER STREET, NIXON PEABODY LLP, BOSTON, MA, 02110-2131, US

(21) Appl. No.: 10/494,141

(22) PCT Filed: Oct. 29, 2002
(86) PCT No.: PCT/CA02/01628

§ 371 Date: Jan. 20, 2005

Related U.S. Application Data
(60) Provisional application No. 60/330,719, filed on Oct. 29, 2001.

(51) Int. Cl. C07H 21/02; C12N 15/85

(52) U.S. Cl. 435/455; 536/23.1; 544/244

(57) ABSTRACT

Oligonucleotides having an internal acyclic linker residue, and the preparation and uses thereof, are described. Such uses include the preparation of acyclic linker-containing antisense oligonucleotides, and their use for the prevention or depletion of function of a target nucleic acid of interest, such as RNA, in a system. Such a prevention or depletion of function includes, for example, the prevention or inhibition of the expression, reverse transcription and/or replication of the target nucleic acid, as well as the cleavage/degradation of the target nucleic acid. Accordingly, an oligonucleotide of the invention is useful for analytical and therapeutic methods and uses in which the function of a target nucleic acid is implicated, as well as a component of commercial packages corresponding to such methods and uses.

### FIELD OF THE INVENTION

The invention relates to modified oligonucleotides and uses thereof, and particularly relates to modified oligonucleotides having one or more acyclic residues at internal positions, and uses thereof.

### BACKGROUND OF THE INVENTION

Oligonucleotides are utilized for a variety of biotechnological applications, including primers, probes, linkers, segments to confer a site or region of interest (e.g. sites for cleavage by nucleases; coding segments), mutagenesis, or to target a particular target region or molecule to fulfill a particular purpose or function. Their ability to confer specificity by virtue of their sequence composition has resulted in their use in a number of applications in biotechnology, in particular cases with various adaptations and modifications to render them more amenable to certain applications. Such modifications may entail the attachment of various groups, or modifications to the individual nucleoside groups or portions (i.e. the sugar and/or the base moieties) thereof, or to the backbone of the oligonucleotide molecule. Given, for example, their ability to be designed to target a protein-encoding molecule, such as RNA, a particular use of oligonucleotides is in antisense technology, to modulate the level, or features of a protein, and in turn modulate the function ascribed to that protein.

Antisense Oligonucleotides (AON)

Antisense oligonucleotides (AONs) have attracted considerable interest in the biotechnology sector, and have exceptional potential for use in therapeutic strategies against a range of human diseases, including cancer and infectious diseases (Uhlmann, E.& Peyman, A. Chem. Rev. 1990, 90, 543). Criteria required of AON for potential clinical use include stability against serum and cellular nucleases, cell-membrane permeability, and stable and specific binding of the AON to its cellular target (usually messenger RNA [mRNA]). The formation of a duplex between the AON and its complementary sequence on the target RNA prevents the translation of such RNA, in part by “translation arrest” (via duplex formation between the AON and, the target RNA, thus inhibiting/preventing complete translation by physically or sterically blocking the translational machinery) but more importantly by eliciting degradation of the targeted RNA through the action of ribonuclease H(RNase H), a ubiquitous and endogenous cellular enzyme that specifically degrades the RNA strand in the AON/RNA duplex (Walder, R. T.; Walder, J. A. Proc. Natl. Acad. Sci. USA 1988, 85, 5011).

Current AON technologies are deficient in one or more of the criteria required for clinical utility. Since the natural substrate of RNase H is a DNA/RNA heteroduplex, DNA has been utilized for antisense technology. However, as serum and intracellular nucleases rapidly degrade AONs with phosphodiester (PDE) linkages, AON consisting of PDE-DNA have had limited utility in such systems. DNA strands with phosphorothioate linkages (PS-DNA) have been used successfully in a large number of experiments designed to downregulate gene expression, and they have been and/or are in use in several clinical therapeutic trials (Akhtar, S. & Agrawal, S. Trends Pharmacol. Sci. 1997, 18, 12). PS-DNA induces RNase H degradation of the targeted RNA, and is resistant to degradation by serum and cellular nucleases, however, it forms weaker duplexes with the target RNA compared to PDE-DNA. Furthermore, PS-DNA shows extensive ‘non-specific’ binding to serum and cellular proteins (Brach, A. D. TIBS, 1998, 23, 45). This can lead to unfavorable toxicity, especially given the high concentrations of PS-DNA needed to exert an in vivo effect. Identification of new AON structures that can bind tightly and specifically to target RNA, and elicit efficient RNase H degradation of that RNA, is a high priority in antisense development.

The structure of the AON determines Whether RNase H can cleave the RNA strand of AON/RNA duplexes. As such, various strategies have been utilized to improve binding to the RNA target, to improve duplex formation and stability. For example, AONs that exclusively contain either 2′-O-methylribose (or any substitution at the ribose 2′-position) or N3′-P5′ phosphoramidate linkages, and DNA molecules containing uncharged internucleotide linkages, composed for example of methylphosphonate or amide linkages, have been described, however, such AON do not elicit RNase H activity (for a review, see Manoharan, M. Biophys. Biochim. Acta, 1999, 1489, 117). Other analogues such as phosphorodiamidate morpholino nucleic acids also lack the ability to elicit RNase H activity (Summerton, J., and Weller, D., Antisense Nucleic Acid Drug Dev., 1997, 7, 187). Peptide nucleic acids (PNA) display remarkable hybridization properties, binding to single stranded RNA, single stranded DNA and duplex DNA with high affinity (Egholm, M. et al., Nature, 1993, 365, 566; Knudsen, H. et al., Nucl. Acids Res., 1997, 25, 2167). However, PNA:RNA hybrids are not substrates for RNase H. In fact, of the several dozens of modified AON prepared during the period 1978-1998, only PS-DNA, phosphorodithioate DNA (PS₂-DNA), and boranophosphate DNA were reported to elicit RNase H degradation of target RNA (Sanghvi, Y. S. & Cook, P. D. “Carbohydrate Modifications in Antisense Research” ACS Symposium Series, vol. 580. American Chemical Society, Washington D.C., 1994). As in the case for PS-DNA, the analogues PS₂-DNA and boranophosphate DNA exhibit weaker binding towards target RNA relative to the unmodified PDE-DNA. Therefore, while the above strategies are capable of conferring increased binding to the target, such AON are unable to induce RNase H activity.

Attempts to overcome this limitation include the development of arabinonucleic acid (ANA) and 2′-deoxy-2′-fluoroarabinonucleic acids (FANA). These compounds are the first sugar-modified oligonucleotides ever reported to elicit RNase H activity [Damha, M. J. et al., “Antisense oligonucleotide constructs based on beta-arabinose and its analogues”. PCT International Publication No. WO 99/67378; Damha, M. J. et al. J. Am. Chem. Soc. 1998, 120, 12976; Noronha, A. M. et al. Biochemistry 2000, 39, 7050). These oligonucleotides retain a β-D-furanose ring and mimic the conformation of DNA strands (Trempe, J.-F. et al., J. Am. Chem. Soc. 2001, 124, 4896). FANA forms much more stable duplexes with target RNA than does PS-DNA; indeed, the stability of the FANA/RNA duplex generally exceeds that of RNA/RNA duplexes (Damha, M. J. et al. J. Am. Chem. Soc. 1998, 120, 12976; Wilds, C. J. & Damha, M. J. Nucl. Acids Res. 2000, 28, 3625).

Other notable developments in the antisense area include mixed-backbone oligonucleotides (MBO) composed of PS-DNA oligodeoxynucleotide segments flanked on both sides by sugar-modified oligonucleotide segments such as PS-[2′-OMe RNA-(DNA)-2′OMe RNA] (for example, see Crooke, S. T. et al., Biochem. J. 1995, 312 (Pt 2), 599). These MBOs are also known as “gapmers”. The flanking 2′-O-methyl RNA “wings” increase the binding affinity of the MBO for target RNA, while the PS-DNA segment in the middle of the AON directs RNase H degradation of the target RNA (Zhao, G. et al., Biochem. Pharmacol. 1996, 51, 173; Crooke, S. T. et al. J. Pharmcol. Exp. Ther. 1996, 277, 923). MBOs have increased stability in vivo (i.e., resistance to nuclease degradation), and show improved biological activity both in vitro and in vivo compared to the corresponding all PS-DNA AON. Examples of this approach incorporating 2′-OMe and other alkoxy substituents in the flanking regions of an oligonucleotide have been demonstrated by Monia et al. by enhanced antitumor activity in vivo (Monia, P. B. et al., Nature Med. 1996, 2, 668). Several pre-clinical trials with these analogues are ongoing (Akhtar, S.; Agrawal, S. TiPS 1997, 18, 12).

MBO antisense comprised of FANA flanking internal DNA segments show exceptionally potent target-specific inhibition of gene expression (EC₅₀<5 nM) when tested in cell culture assays, and unlike 2′-OMe RNA/DNA MBO, their biological activity is significantly less dependent on the length of the internal DNA gap (Damha et al.; International PCT Publication WO 02/20773 published Mar. 14, 2002).

Elicitation of Cellular RNase H Degradation of Target RNA by AONs

RNase H selectively degrades the RNA strand of a DNA/RNA heteroduplex (Hausen, P.; Stein, H. Eur. J. Biochem. 1970, 14, 279). One of the most important mechanisms for antisense oligonucleotide-directed inhibition of gene expression is the ability of these antisense oligonucleotides to form a structure, when duplexed with the target RNA, that can be recognized by cellular RNase H. This enables the RNase, H-mediated degradation of the RNA target, within the region of the antisense oligonucleotide-RNA base-paired duplex (Walder, R. T.; Walder, J. A. Proc. Natl. Acad. Sci. USA 1988, 85, 5011).

RNase H1 from the bacterium Escherichia coli is the most readily available and the best characterized enzyme. Studies with eukaryotic cell extracts containing RNase H suggest that both prokaryotic and eukaryotic enzymes exhibit similar RNA-cleavage properties (Monia et al. J. Biol. Chem. 1993, 268, 14514; Crooke et al. Biochem J. 1995, 312, 599; Lima, W. F.; Crooke, S. T. Biochemistry 1997, 36, 390). E. coli RNase H1 is thought to bind to the minor groove of the DNA/RNA double helix and to cleave the RNA by both endonuclease and processive 3′-to-5′ exonuclease activities (Nakamura, H. et al. Proc. Natl. Acad. Sci. USA 1991, 88, 11535; Fedoroff, O. Y. et al., J. Mol. Biol. 1993, 233, 509). The efficiency of RNase H degradation displays minimal sequence dependence and, as mentioned above, is quite sensitive to chemical changes in the antisense oligonucleotide.

Because 2′-OMe RNA cannot elicit RNase H activity, the DNA gap size of the PS-[2′-OMe RNA-DNA-2′OMe RNA] chimeric oligonucleotides must be carefully defined. Thus, while E. coli RNase H can recognize and use 2′-OMe RNA MBO with DNA gaps as small as 4 DNA nucleotides (Shen, L. X. et al. Bioorg. Med. Chem. 1998, 6, 1695), the eukaryotic RNase H (such as human RNase HII) requires larger DNA gaps (7 DNA nucleotides or more) for optimal degradative activity (Monia, B. P. et al. J. Biol. Chem. 1993, 268, 14514). In general, with PS-[2′-OMe RNA-DNA-2′OMe RNA] chimeric oligonucleotides, eukaryotic RNase H-mediated target RNA cleavage efficiency decreases with decreasing DNA gap length, and becomes almost negligible with DNA gap sizes of less than 6 DNA nucleotides. Thus, the antisense activity of PS-[2′-OMe RNA-DNA-2′OMe RNA] chimera oligonucleotides is highly dependent on DNA gap size (Monia, B. P. et al. J. Biol. Chem. 1993, 268, 14514; Agrawal, S. and Kandimalla, E. R. Mol. Med. Today 2000, 6, 72). This is not the case for PS-[FANA-DNA-FANA] chimeras which display significant biological activity with DNA gaps as small as 1 deoxynucleotide residue (Damha et al.; International PCT Publication WO 02/20773 published Mar. 14, 2002).

Recently, oligonucleotides containing completely altered backbones have been synthesized. Notable examples are the peptide nucleic acids (“PNA”) with an acyclic backbone (Nielsen, P. E. in “Perspectives in Drug Discovery and Design”, vol. 4, pp. 76, Trainor, G. L. (ed.), ESCOM, Leiden, 1996). These compounds have exceptional hybridization properties, and stability towards nucleases and proteases. However, efforts to use PNA oligomers as antisense constructs have been hampered by poor cellular uptake and inability to activate RNase H. Very recently, PNA-[DNA]-PNA chimeras have been designed to maintain RNase H mediated cleavage via the DNA portion of the chimera (Bergman, F. et al., Tetrahedron Lett. 1995, 36, 6823; Van der Laan, A. C. et al. Trav. Chim. Pays-Bas 1995, 114, 295). The PNA segments located at the 5′- and 3′-termini serve to facilitate binding to the target nucleic acid (RNA) and enhance resistance towards degradation by exonuclease enzymes. However, based on the presence of DNA, such a construct may be more prone to degradation in biological systems, as noted above.

There is therefore a need for an improved oligonucleotide for such antisense approaches, to try to address the limitations noted above (e.g. binding, induction of RNase H activity, resistance to degradation).

### SUMMARY OF THE INVENTION

According to an aspect of the invention, there is provided an oligonucleotide having the structure:
[R¹—X]ₐ—R² Ia
wherein a is greater than or equal to 1; wherein each of R¹ and R² are independently at least one nucleotide; and wherein X is an acyclic linker. In an embodiment, the oligonucleotide comprises at least one modified deoxyribonucleotide, i.e. either R¹, R² or both may comprise at least one modified deoxyribonucleotide.

In an embodiment, the modified deoxyribonucleotide is selected from the group consisting of ANA, PS-ANA, PS-DNA, RNA-DNA and DNA-RNA chimeras, PS-[RNA-DNA] and PS-[DNA-RNA] chimeras, PS-[ANA-DNA] and PS-[DNA-ANA] chimeras, RNA, PS-RNA, PDE- or PS-RNA analogues, locked nucleic acids (LNA), phosphorodiamidate morpholino nucleic acids, N3′-P5′ phosphoramidate DNA, cyclohexene nucleic acid, alpha-L-LNA, boranophosphate DNA, methylphosphonate DNA, and combinations thereof. In an embodiment, the ANA is FANA (e.g. PDE- or PS-FANA).

In an embodiment, the above-mentioned PDE- or PS-RNA analogues are selected from the group consisting of 2′-modified RNA wherein the 2′-substituent is selected from the group consisting of alkyl, alkoxy, alkylalkoxy, F and combinations thereof.

In an embodiment, the acyclic linker is selected from the group consisting of an acyclic nucleoside and a non-nucleotidic linker. In embodiments, the acyclic nucleoside is selected from the group consisting of purine and pyrimidine seconucleosides. In embodiments, the purine seconucleoside is selected from the group consisting of secoadenosine and secoguanosine. In embodiments, the pyrimidine seconucleoside is selected from the group consisting of secothymidine, secocytidine and secouridine.

In an embodiment, the non-nucleotidic linker comprises a linker selected from the group consisting of an amino acid and an amino acid derivative. In embodiments, the amino acid derivative is selected from the group consisting of (a) an N-(2-aminoethyl)glycine unit in which an heterocyclic base is attached via a methylene carbonyl linker (PNA monomer); and (b) an O-PNA unit.

According to a further aspect of the invention, there is provided an AON chimera of general structure Ib: 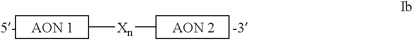
wherein n is greater than or equal to 1. With reference to structure lb above, “AON1” is an oligonucleotide chain, which in embodiments is selected from the group consisting of ANA (e.g. FANA), DNA, PS-DNA, 5′-RNA-DNA-3′ chimeras, as well as other RNase H-competent oligonucleotides, for example arabinonucleic acids (2′-OH substituted ANA) (Damha, M. J. et al. J. Am. Chem. Soc. 1998, 120, 12976), cyclohexene nucleic acids (Wang J. et al. J. Am. Chem. Soc. 2000, 122, 8595), boranophosphate linked DNA (Rait, V. K. et al. Antisense Nucleic Acid Drug Dev. 1999, 9, 53), and alpha-L-locked nucleic acids (Sørensen, M. D. et al. J. Am. Chem. Soc. 2002, 124, 2164) or combinations thereof; and “AON2” is an oligonucleotide chain, which in embodiments is selected from the group consisting of FANA, DNA, PS-DNA, 5′-DNA-RNA-3′ chimeras, as well as other RNase H-competent oligonucleotides such as those described above, or combinations thereof. The internucleotide linkages of the AON1 and AON2 includes but is not necessarily limited to phosphodiester, phosphotriester, phosphorothioate, methylphosphonate, phosphoramidate (5′N-3′P and 5′P-3′N) groups. The substituent directly attached to the C2′-atom of the arabinose sugar in ANA-X-ANA chimera constructs includes but is not limited to fluorine, hydroxyl, amino, azido, alkyl (e.g. 2′-methyl, ethyl, propyl, butyl, etc.), and alkoxy groups (e.g., 2′-OMe, 2′-OEt, 2′-OPr, 2′-QBu, 2′-OCH₂CH₂OMe, etc.).

Examples of the general structures Ia and Ib include PDE- and PS-[FANA]-X-[FANA], PDE- and PS-[FANA-DNA-X-DNA-FANA], PS-[DNA-X-DNA], PDE- and PS-[RNA-DNA-X-DNA-RNA], PDE- and PS-[(2′O-alkyl-RNA)-DNA-X-DNA-(2′O-alkyl-RNA)], and PDE- and PS-[(2′-OCH₂CH₂OMe-RNA)-DNA-X-DNA-(2′-OCH₂CH₂OMe-RNA)].

In an embodiment, an oligonucleotide of the invention has the structure: 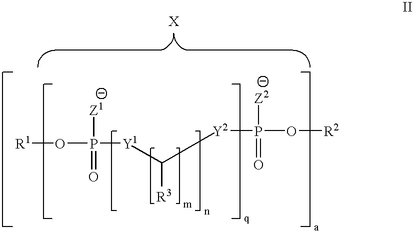
wherein each of m, n, q and a are independently integers greater than or equal to 1; wherein each of R¹ and R² are independently at least one nucleotide, wherein each of Z¹ and Z² are independently selected from the group consisting of an oxygen atom, a sulfur atom, an amino group and an alkylamino group;
wherein each of Y¹ and Y² are independently selected from the group consisting of oxygen, sulfur and NH; and wherein R³ is selected from the group consisting of H, alkyl, hydroxyalkyl, alkoxy, a purine, a pyrimidine and combinations thereof.

In embodiments, R³ is adenine or guanine, or derivatives thereof.

In embodiments, R³ is thymine, cytosine, 5-methylcytosine, uracil, or derivatives thereof.

In embodiments, each of R¹ and R² noted above are independently selected from the group consisting of ANA, PS-ANA, PS-DNA, RNA-DNA and DNA-RNA chimeras, PS-[RNA-DNA] and PS-[DNA-RNA] chimeras, PS-[ANA-DNA] and PS-[DNA-ANA] chimeras, alpha-L-LNA, cyclohexene nucleic acids, RNA, PS-RNA, PDE- or PS-RNA analogues, locked nucleic acids (LNA), phosphorodiamidate morpholino nucleic acids, N3′-P5′ phosphoramidate DNA, methylphosphonate DNA, and combinations thereof.

In embodiments, each of R¹ and R² noted above independently may comprise at least two nucleotides connected via an internucleotide linkage, wherein said internucleotide linkage is selected from the group consisting of phosphodiester, phosphotriester, phosphorothioate, methylphosphonate, phosphoramidate (5′N-3′P and 5′P-3′N) groups and combinations thereof.

In embodiments, each of R¹ and R² noted above independently comprise ANA.

In embodiments the above-noted ANA comprises a 2′-substituent selected from the group consisting of fluorine, hydroxyl, amino, azido, alkyl (e.g. methyl, ethyl, propyl and butyl) and alkoxy (e.g. methoxy, ethoxy, propoxy, and methoxyethoxy) groups.

In an embodiment, the 2′-substituent is fluorine and said ANA is FANA.

In embodiments, the alkyl group is selected from the group consisting of methyl, ethyl, propyl and butyl groups.

In embodiments, the alkoxy group is selected from the group consisting of methoxy, ethoxy, propoxy, and methoxyethoxy groups.

In embodiments, an oligonucleotide of the invention is selected from the group consisting of: 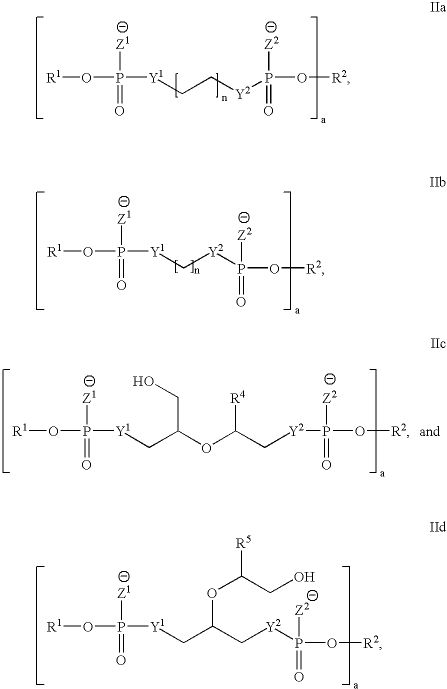
wherein R¹, R², n, a, Z¹, Z², Y¹ and Y² are as defined above and each of R⁴ and R⁵ are independently selected from the group consisting of a purine (e.g. adenine and guanine or derviatives thereof) and a pyrimidine (e.g. thymine, cytosine, uracil, or derivatives thereof).

In an embodiment, R¹ and R² are PDE-FANA; and a=1.

In an embodiment, R¹ and R² are PS-FANA; and a=1.

In an embodiment, R¹ is [FANA-DNA]; R² is [DNA-FANA]; and a=1.

In an embodiment, R¹ is [FANA-DNA]; R² is FANA; and a=1.

In an embodiment, R¹ is FANA; R² is [DNA-FANA]; and a=1.

In an embodiment, R¹ and R² are PS-DNA; and a=1.

In an embodiment, R¹ is PDE-[RNA-DNA], R² is PDE-[DNA-RNA]; and a=1.

In an embodiment, R¹ is RNA; R² is [DNA-RNA]; and a=1.

In an embodiment, R¹ is S-[(2′O-alkyl)RNA-DNA]; R² is S-[DNA-(2′O-alkyl)RNA]; and a=1.

In an embodiment, R¹ is S-[(2′O-alkyl)RNA-DNA]; R² is S-[(2′O-alkyl)RNA); and a=1.

In an embodiment, R¹ is S-[(2′O-alkyl)RNA]; R² is S-[DNA-(2′O-alkyl)RNA]; and a=1.

In an embodiment, R¹ is S-[(2′O-alkoxyalkyl)RNA-DNA]; R² is S-[DNA-(2′O-alkoxyalkyl)RNA]; and a=1.

In an embodiment, R¹ is S-[(2′O-alkoxyalkyl)RNA-DNA]; R² is S-[(2′O-alkoxyalkyl)RNA]; and a=1.

In an embodiment, R¹ is S-[(2′O-alkoxyalkyl)RNA]; R² is S-[DNA-(2′O-alkoxyalkyl)RNA]; and a=1.

In an embodiment, R¹ is PDE-[(2′O-alkyl-RNA)-DNA]; R² is PDE-[DNA-(2′O-alkyl RNA)]; and a=1.

In an embodiment, R¹ is PS-[(2′O-alkyl-RNA)-DNA]; R² is PS-[DNA-(2′O-alkyl RNA)]; and a=1.

In an embodiment, R¹ is PDE-[(2′O-alkoxyalkyl-RNA)-DNA]; R² is PDE-[DNA-(2′O-alkoxyalkyl RNA)]; and a=1.

In an embodiment, R¹ is PS-[(2′O-alkoxyalkyl-RNA)-DNA]; R² is PS-[DNA-(2′O-alkoxyalkyl RNA)]; and a=1.

In an embodiment, R¹ and R² are PDE-[FANA]; a=1; and the oligonucleotide has structure IIb in which Y¹, Y² are oxygen; Z¹, Z² are both oxygen or sulfur, and n=4.

In an embodiment, R¹ is PS-[FANA]; R² is PDE-[FANA]; a=1; and the oligonucleotide has structure IIb in which Y¹, Y² are oxygen; Z¹, Z² are both oxygen or sulfur, and n=4.

In an embodiment, R¹ is FANA; R² is PS-FANA; a=1; and the oligonucleotide has structure IIb in which Y¹, Y², Z¹ and Z² are oxygen and n=4.

In an embodiment, R¹ and R² are PS-[FANA]; a=1; and the oligonucleotide has structure IIb in which Y¹, Y² are oxygen; Z¹, Z² are both oxygen or sulfur, and n=4.

In an embodiment, R¹ is PS-[DNA]; R² is PDE-[DNA]; a=1; and the oligonucleotide has structure IIb in which Y¹, Y² are oxygen; Z¹, Z² are both oxygen or sulfur, and n=4.

In an embodiment, R¹ is PDE-[DNA]; R² is PS-[DNA]; a=1; and the oligonucleotide has structure IIb in which Y¹, Y² are oxygen; Z¹, Z² are both oxygen or sulfur, and n=4.

In an embodiment, R¹ and R² are PS-[DNA]; a=1; and the oligonucleotide has structure IIb in which Y¹, Y² are oxygen; Z¹, Z² are both oxygen or sulfur, and n=4.

In an embodiment, R¹ and R² are PDE-[FANA]; a=1; and the oligonucleotide has structure IIc in which Y¹, Y² are oxygen; Z¹, Z² are both oxygen or sulfur.

In an embodiment, R¹ is PS-[FANA]; R² is PDE-[FANA]; a=1; and the oligonucleotide has structure IIc in which Y¹, Y² are oxygen; Z¹, Z² are both oxygen or sulfur.

In an embodiment, R¹ is PDE-[FANA]; R² is PS-[FANA]; a=1; and the oligonucleotide has structure IIb in which Y¹, Y² are oxygen; Z¹, Z² are both oxygen or sulfur, and n=4.

In an embodiment, R¹ and R² are PS-[FANA]; a=1; and the oligonucleotide has structure IIc in which Y¹, Y² are oxygen; Z¹, Z² are both oxygen or sulfur.

In an embodiment, R¹ is PS-[DNA]; R² is PDE-[DNA]; a=1; and the oligonucleotide has structure IIc in which Y¹, Y² are oxygen; Z¹, Z² are both oxygen or sulfur.

In an embodiment, R¹ is DNA; R² is PS-DNA; a=1; and the oligonucleotide has structure IIc.

In an embodiment, R¹ and R² are PS-[DNA]; a=1; and the oligonucleotide has structure IIc in which Y¹, Y² are oxygen; Z¹, Z² are both oxygen or sulfur.

In an embodiment, a=2 and each of R¹ and R² independently consist of at least 3 nucleotides, in a further embodiment, of 3-8 nucleotides.

In an embodiment, a=3 and each of R¹ and R² independently consist of at least 2 nucleotides, in a further embodiment, wherein each of R¹ and R² independently consist of 2-6 nucleotides.

In an embodiment, the oligonucleotide is antisense to a target RNA.

The invention further provides a method of preventing or decreasing translation, reverse transcription and/or replication of a target RNA in a system, said method comprising contacting said target RNA with an oligonucleotide as defined above.

The invention further provides a method of preventing or decreasing translation, reverse transcription and/or replication of a target RNA in a system, said method comprising:
a) contacting said target RNA with an oligonucleotide as defined above; and
b) allowing RNase cleavage of said target RNA.

The invention further provides a use of an oligonucleotide as defined above for preventing or decreasing translation, reverse transcription and/or replication of a target RNA in a system.

The invention further provides a commercial package comprising the above-noted oligonucleotide together with instructions for its use in preventing or decreasing translation, reverse transcription and/or replication of a target RNA in a system.

### BRIEF DESCRIPTION OF THE DRAWINGS

The invention will now be described in greater detail having regard to the appended drawings in which:

FIG. 1 illustrates RNase H mediated cleavage of RNA duplexed with homopolymeric PDE-FANA and FANA-X-FANA. Timed aliquots were taken at 0, 5, 10, and 20 min from each set of incubation. Experimental conditions are given in Example 4A.

FIG. 2 illustrates RNase H mediated cleavage of RNA duplexed with homopolymeric PDE-FANA and PDE-[FANA-X-FANA] as a function of time. Degradation of the 5′-labeled target RNA was quantified by densitometry of the gel shown in FIG. 1.

FIG. 3 illustrates RNase H mediated cleavage of RNA duplexed with mixed-base PDE-FANA and PDE-[FANA-X-FANA]. Timed aliquots were taken at 0, 5, 10, and 20 min from each set of incubation. Experimental conditions are given in Example 4B.

FIG. 4 illustrates RNase H mediated cleavage of RNA duplexed with mixed base PDE-FANA and PDE-[FANA-X-FANA] as a function of time. Degradation of the 5′-labeled target RNA was quantified by densitometry of the gel shown in FIG. 3.

FIG. 5 illustrates RNase H mediated cleavage of RNA duplexed with homopolymeric PDE-FANA-X-FANA] containing the butanediol linker X at positions 5, 10 and 13. Assays (10 μL final volume) comprised 1 pmol of 5′-[³²P]-target RNA and 3 pmol of test oligonucleotide in 60 mM Tris-HCl (pH 7.8, containing 2 mM dithiothreitol, 60 mM KCl, and 10 mM MgCl₂. Reactions were started by the addition of RNase H and carried out at 14-15° C. for 20 minutes. Timed aliquots were taken at 0, 5, 10, and 20 min from each set of incubation. Lengths of the RNA fragments generated via enzyme scission and corresponding position along the AON are indicated.

FIG. 6 illustrates RNase H mediated cleavage of RNA duplexed with homopolymeric FANA-X-FANA (But-5, 10 and 13) as a function of time. Degradation of the 5′-labeled target RNA was quantified by densitometry of the gel shown in FIG. 5.

FIG. 7 illustrates RNase H mediated cleavage of RNA duplexed with homopolymeric PDE-[FANA-X-FANA] and PDE-[FANA-X-X-FANA] containing internal secouridine linkers. Timed aliquots were taken at 0, 5, 10, and 20 min from each set of incubation. Experimental conditions are given in Example 6.

FIG. 8 illustrates RNase H mediated cleavage of RNA duplexed with homopolymeric PDE-[FANA-X-FANA] (SEC×1), and PDE-[FANA-X-X-FANA] (SEC×2), and PDE-FANA as a function of time. Degradation of the 5′-labeled target RNA was quantified by densitometry of the gel shown in FIG. 7.

FIG. 9 illustrates RNase H mediated cleavage of RNA duplexed with homopolymeric PDE-DNA and PDE-[DNA-X-DNA] (X=butanediol linker). Timed aliquots were taken at 0, 5, 10, and 20 min from each set of incubation. Experimental conditions are given in Example 7.

FIG. 10 illustrates RNase H mediated cleavage of RNA duplexed with homopolymeric PDE-DNA and PDE-[DNA-X-DNA] (X=butanediol linker) as a function of time. Degradation of the 5′-labeled target RNA was quantified by densitometry of the gel shown in FIG. 9.

FIG. 11 illustrates RNase H mediated cleavage of Ha-Ras RNA duplexed with mixed base PDE-FANA, PDE-[FANA-X-FANA], PDE-DNA, PDE-[DNA-X-DNA], and PDE-[mismatched DNA] containing the butanediol linker X at position 10. Assays were conducted as described in Example 8. Lengths of the RNA fragments generated via enzyme scission and corresponding position along the AON are indicated. Kinetic data (k) of RNA cleavage is provided in Table 1.

FIG. 12 illustrates RNase H mediated cleavage of Ha-Ras RNA duplexed with mixed base PS-FANA, PS-[FANA-X-FANA], PS-DNA, and PS-[DNA-X-DNA] containing the butanediol linker X at position 10. Assays were conducted as described in Example 9. Kinetic data (k) of RNA cleavage is provided in Table 1.

### DETAILED DESCRIPTION OF THE INVENTION

The invention relates to modified oligonucleotides that, in an embodiment, are capable of selectively preventing gene expression in a sequence-specific manner. In particular, the invention relates to the selective inhibition of protein biosynthesis via antisense strategy using short strands of for example modified nucleotides, such as modified DNA and modified arabinonucleic acids, containing one or more acyclic residues at internal positions. In a preferred embodiment, an oligonucleotide of the invention comprises at least one modified nucleoside or nucleotide (as compared to native DNA). Examples of acyclic residues include acyclic nucleosides [e.g., seconucleosides, PNA monomers (N-(2-aminoethyl)glycine unit in which a heterocyclic base is attached via a methylene carbonyl linker), O-PNA monomers [—NH—CH(CH₂—CH₂-Base)-CH₂—O—CH₂—CO—] and non-nucleotidic linkers (e.g., alkyldiol linker, amino acids, dipeptides and dipeptide derivatives). In embodiments the invention relates to the use of modified oligonucleotides constructed primarily from modified deoxyribonucleotide and modified arabinonucleotide residues containing one or more acyclic residues, to hybridize to complementary RNA such as cellular messenger RNA, viral RNA, etc. In a further embodiment, the invention relates to the use of modified oligonucleotides constructed from modified DNA and modified ANA residues, containing one or more acyclic residues, to hybridize to and induce cleavage of complementary RNA via RNase H activation.

In an embodiment, the invention relates to antisense oligonucleotide chimeras constructed from either modified deoxyribonucleotide or modified arabinonucleotide residues flanking an acyclonucleotide or a modified hydrocarbon chain, that form a duplex with its target RNA sequence. The resulting AON/RNA duplexes are excellent substrates for RNase H, an enzyme that recognizes this duplex and degrades the RNA target portion. RNase H-mediated cleavage of RNA targets is considered to be a major mechanism of action of antisense oligonucleotides.

The present invention relates to the unexpected and surprising discovery that antisense chimeras constructed from a modified nucleotide (e.g. 2′-deoxy-2′-fluoro-β-D-arabinonucleotides [FANA]) and an internal acyclic nucleotide residue (e.g. seconucleotide), or an internal modified hydrocarbon chain are superior at eliciting eukaryotic RNase H activity in vitro compared to uniformly modified FANA oligomers. Accordingly, antisense hybrid chimeras comprising a modified nucleotide such as 2′-deoxy-2′-fluoro-β-D-arabinonucleotides (FANA), containing such RNase H-inducing acyclic residues may be useful as therapeutic agents and/or tools for the study and control of specific gene expression in cells and organisms. This “acyclic linker strategy” may also be applied to other modified AONs in order to improve their antisense properties in vivo.

The results described herein are truly surprising based on the current wisdom in the art, because a consistent and prevailing goal in antisense technology has always been to introduce modifications that increase duplex stability. Such modifications in many cases result in a type of “pre-organization” of the antisense molecule, whereby the AON is designed to resemble the “bound” conformation even before duplex formation occurs, thus reducing the entropy associated with binding. As such, introduction of a flexible structural element such as an acyclic linker (which is free of the ring strain of a cyclic structure), since it would decrease duplex stability, is considered to be detrimental to RNase H induction. Indeed, the introduction of such acyclic elements results in a lower melting temperature as outlined in the results presented herein. Consistent with this principle, native DNA oligonucleotides bridged by oligomethylenediol or oligoethylene glycol linkers have been described as exhibiting decreased duplex stability and impaired RNase H activity (Vorobjev et al., Antisense and Nucleic Acid Drug Dev., 2001, 11, 77).

Conversely, applicants' studies described herein demonstrate that the incorporation of flexible structural elements such as an acyclic linker in for example 2′F-ANA AON results in efficient RNase H-mediated target cleavage. It is shown herein that the enzyme's activity is readily modulated by the systematic placement of flexible units at key sites within for example 2′F-ANA strands of AON/RNA duplexes. Based on the improved induction of RNase H using AON comprising modified nucleotides described herein, it is envisioned that a certain amount of pre-organization (e.g. conferred by including one or more modified nucleotides in the oligonucleotide) in the antisense strand plays a role in maintaining high binding and/or specificity for complementary RNA. While both pre-organization & flexibility on their own are detrimental towards enzyme elicitation, applicants propose herein the surprising finding that their combination gives synergistic inhibition of target mRNA and address the various conformational characteristics that give rise to these enhancements. As such, applicants describe herein that even compounds devoid of DNA can elicit RNase H activity with comparable efficiency to the native (DNA) systems by virtue of an introduced acyclic linker. Further, the improved induction of RNase H conferred by such an acyclic linker is even more pronounced when targeting longer (i.e. more physiologically relevant) RNAs, as described herein. Therefore, it is envisioned that such an acyclic linker strategy may be incorporated into known antisense methodologies and structures to improve RNase H induction and in turn target inhibition.

“Flexible” or “flexibility” as used herein is a relative term referring to the degrees of freedom with respect to allowable motion or conformations available at a particular region of interest in a molecule, thus contributing to the “flexibility” of the molecule overall. As such, a flexible element is one which is introduced into a region where prior to its addition more rigid elements were present. In embodiments, a flexible element in an oligonucleotide is an acyclic linker, which is more flexible than a cyclic backbone structure due to the absence of ring strain as compared to the cyclic structure.

According to an aspect of the invention, there is provided an oligonucleotide of the structure Ia:
[R¹—X]ₐ—R² IA
wherein a is greater than or equal to 1, each of R¹ and R² are independently at least one nucleotide and
X is an acyclic linker.

According to a further aspect of the invention there is provided an AON chimera of general structure Ib: 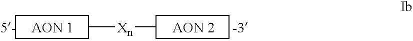
wherein n is greater than or equal to 1. With reference to structure Ib above, “AON1” is an oligonucleotide chain, which in embodiments is selected from the group consisting of FANA, DNA, S-DNA, and 5′-RNA-DNA-3′ chimeras and combinations thereof; and “AON2” is an oligonucleotide chain, which in embodiments is selected from the group consisting of FANA, DNA, S-DNA, and 5′-DNA-RNA-3′ chimeras and combinations thereof. The internucleotide linkages of the AON1 and AON2 include but are not necessarily limited to phosphodiester, phosphotriester, phosphorothioate, methylphosphonate, and phosphoramidate (5′N-3′P and 5′P-3′N) groups. The 2′-substituent of the arabinose sugar in ANA-containing constructs includes but is not limited to fluorine, hydroxyl, amino, azido, methyl, methoxy and other alkoxy groups (e.g., ethoxy, propoxy, methoxyethoxy, etc.).

Examples of the general structures Ia and Ib include phosphodiester linked FANA-X-FANA, RNA-DNA-X-DNA-RNA, (2′O-alkyl-)RNA-DNA-X-DNA-(2′O-alkyl)RNA, (2′-alkylalkoxy)RNA-DNA-X-DNA-(2′O-alkylalkoxy)RNA, and the corresponding phosphorothioate linked derivatives. Any of the above structures may comprise DNA. In a preferred embodiment, an oligonucleotide of the invention comprises at least one modified nucleotide, in an embodiment a modified deoxyribonucleotide.

“Acyclic” as used herein, with reference to linkers, refers to a linking backbone structure that does not have a cyclic portion. This feature relates to the backbone structure only, e.g. the backbone structure of an acyclic linker may have a branch or substituent extending therefrom comprising a cyclic group. An acyclic linker which links two nucleotides refers to a linker having a non-cyclic backbone structure joining the two nucleotides.

“Modified nucleotide/nucleoside” as used herein refers to a nucleotide/nucleoside that differs from and thus excludes the defined native form. For example, a modified deoxyribonucleotide is a molecule other than native DNA. Further, by such definition, a modified deoxyribonucleotide encompasses native RNA. Modifications may comprises additions, deletions or substitutions at one or more parts of a molecule, e.g. at the base, sugar phosphate and/or backbone portions.

“Nucleoside” refers to a base (e.g. a purine [e.g. A and G] or pyrimidine [e.g. C, 5-methyl-C, T and U]) combined with a sugar (e.g. [deoxy]ribose, arabinose and derivatives). “Nucleotide” refers to a nucleoside having a phosphate group attached to its sugar moiety. In embodiments these structures may include various modifications, e.g. either in the base, sugar and/or phosphate moieties. “Oligonucleotide” as used herein refers to a sequence comprising a plurality of nucleotides joined together. An oligonucleotide may comprise modified structures in its backbone structure and/or in one or more of its component nucleotides. In embodiments, oligonucleotides of the invention are about 1 to 200 bases in length, in further embodiments from about 5 to about 50 bases, from about 8 to about 40 bases, and yet further embodiments, from about 12 to about 25 bases in length.

“Alkyl” refers to straight and branched chain saturated hydrocarbon groups (e.g. methyl, ethyl, propyl, butyl, isopropyl etc.). “Alkenyl” and “alkynyl” refer to hydrocarbon groups having at least one C—C double and one C—C triple bond, respectively. “Alkoxy” refers to an —O-alkyl structure. “Alkylamino” refers to —NH(alkyl) or —N(alkyl)₂ structures. “Aryl” refers to substituted and unsubstituted aromatic cyclic structures (e.g. phenyl, naphthyl, anthracyl, phenanthryl, pyrenyl, and xylyl groups). “Hetero” refers to an atom other than C; including but not limited to N, O, or S. In embodiments, the above-mentioned groups may be substituted.

In embodiments, an oligonucleotide of the invention has the structure II: 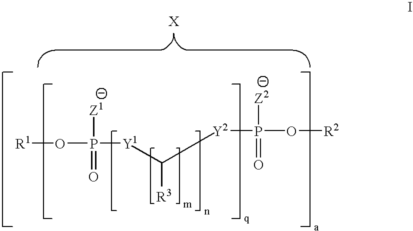
wherein m, n, and q are greater than or equal to 1, P¹ and P² are phosphorus atoms of phosphate groups which are linked to R¹ and R², respectively, each of Z¹ and Z² are independently selected from the group consisting of an oxygen atom, a sulfur atom, an amino group and an alkylamino group, each of Y¹ and Y² are independently selected from the group consisting of oxygen, sulfur and NH; and R³ is selected from the group consisting of H, alkyl, hydroxyalkyl, alkoxy, a “base” (including but not limited to a purine or a pyrimidine) and combinations thereof. In embodiments, the above-noted purine includes adenine and guanine and the above-noted pyrimidine includes thymine, cytosine and uracil. In embodiments, each of R¹ and R² noted above are selected from the group consisting of ANA, DNA, S-DNA, and 5′-DNA-RNA-3′ chimeras or combinations thereof. In embodiments, the above-noted ANA comprises a 2′-substituent selected from the group consisting of fluorine, hydroxyl, amino, azido, alkyl (e.g. methyl, ethyl, propyl and butyl), alkylamino (e.g., propylamino), alkenyl (e.g., —CH═CH₂), alkynyl (e.g., —C≡CH), and alkoxy (e.g. methoxy, ethoxy, propoxy, and methoxyethoxy) groups. When the 2′-substituent is fluorine, the ANA is FANA. In embodiments, R¹ and/or R² comprise at least two nucleotides having at least one internucleotide linkage. In embodiments, the internucleotide linkage is selected from the group consisting of phosphodiester, phosphotriester, phosphorothioate, methylphosphonate, phosphoramidate (5′N-3′P and 5′P-3′N) groups and combinations thereof.

In certain embodiments, an oligonucleotide of the invention is selected from the group consisting of the compounds as set forth in structures IIa, IIb, IIc and IId given below: 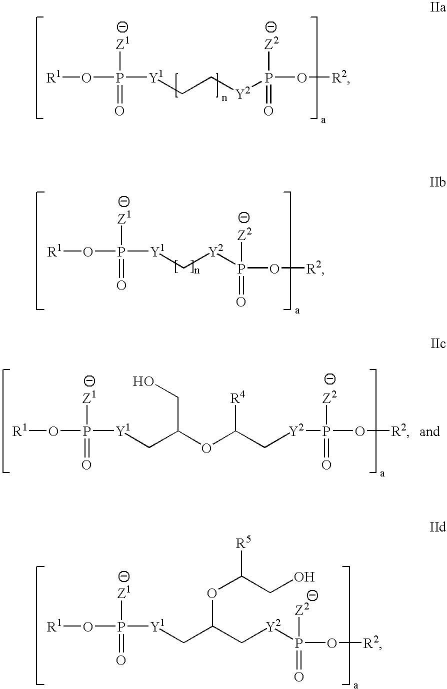
wherein R¹, R², n, a, Z¹, Z², Y¹ and Y² are as defined above. In embodiments, each of R⁴ and R⁵ are independently selected from the group consisting of a “base”, which in embodiments includes but is not limited to a purine or a pyrimidine, examples of which are noted above.

In embodiments, oligonucleotides of the invention are those having the structure FANA-X-FANA, where, in embodiments, X is located at or near the middle of the oligonucleotide sequence, and the oligonucleotide has structure IIb (Y¹=Y²=Z¹=Z²=oxygen, and n=4) or structure IIc (Y¹=Y²=Z¹=Z²=oxygen, and a=1).

According to a further aspect of the invention, there are provided oligonucleotides of the general formula V:

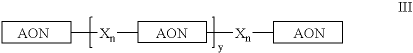

With reference to structure III above, each of y and n are independently an integer greater than or equal to 1; linker X is defined as described above. In embodiments, the oligonucleotide backbone in the definition of AON is selected from the group consisting of ANA (e.g. FANA), DNA, and PS-DNA, and other RNase H competent backbones such as alpha-L-LNA, cyclohexene nucleic acids, or combinations thereof. In embodiments, the internucleotide linkages of the AON includes but is not necessarily limited to phosphodiester, phosphotriester, phosphorothioate, methylphosphonate, phosphoramidate (5′N-3′P and 5′P-3′N) groups. The 2′-substituent of the arabinose sugar when the AON segment is ANA includes but is not limited to fluorine (i.e. FANA), hydroxyl, amino, azido, alkyl (e.g. methyl, ethyl, propyl, butyl, etc.), alkylamino (e.g., propylamino), alkenyl (e.g., —CH═CH₂), alkynyl (e.g., —C≡CH), methoxy and other alkoxy groups (e.g., ethoxy, propoxy, methoxyethoxy, etc.). In embodiments the AON includes but is not necessarily limited to PS-RNA, PDE- or PS-RNA analogues (e.g., 2′-modified RNA in which the 2′-substituent comprises alkyl, 2′-alkoxy, 2′-alkylalkoxy, or 2′-F), locked nucleic acids (LNA), phosphorodiamidate morpholino nucleic acids, N3′-P5′ phosphoramidate DNA, methylphosphonate DNA, and combinations thereof. In certain embodiments, examples of these oligonucleotides include: 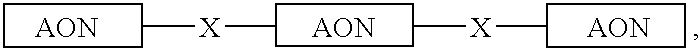
where, in an embodiment, AON is 3-8 nt in length; and 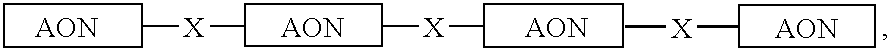
where, in an embodiment, AON is 2-6 nt in length

It will be understood that other structures for the X linkers can be considered, e.g., biodegradable acyclic residues, and acyclic residues containing two types of monomers linked together by for example peptide bonds. Examples include but are not limited to the dipeptide glycine-glycine, and any combination of the naturally occurring amino acids or derivatives thereof. In embodiments, X is an N-(2-aminoethyl)glycine unit in which an heterocyclic base is attached via a methylene carbonyl linker. Other related acyclic peptide monomers may be considered, for example, the O-PNA monomers [—NH—CH(CH₂—CH₂-Base)-CH₂—O—CH₂—CO—] described by Kuwahara et al., J. Am. Chem. Soc. 2001, 123, 4356.

In the case of a PNA-based acyclic linker, the 3′ flanking group may have an amino group at its 5′ terminus, which is linked to the acyclic (X) linker via an amide bond. Other acyclic linkers such as spermine and derivatives, as well as ethylene glycols (e.g. polyethylene glycol or PEG) and derivatives can be considered.

In various embodiments, the oligonucleotide may be designed such that the acyclic linker may or may not “loop out” when the oligonucleotide forms a duplex with its target molecule. “Loop out” as used herein refers to the case where the linker does not itself occupy a position in the oligonucleotide corresponding to a position in the target molecule, effectively forming a loop from the duplex once formed. In the case where the linker does not “loop out”, it occupies a position in the duplex corresponding to a position in the bound target molecule.

The AONs of this invention contain a sequence that is complementary (in certain embodiments partially complementary, and in other embodiments exactly complementary) to a “target RNA”, based on hybridization. “Hybridization” as used herein refers to hydrogen bonding between complementary nucleotides. The degree of complementarity between an AON and its target sequence may be variable, and in embodiments the AON is exactly complementary to its target sequence as noted above. It is understood that it is not essential that an AON be exactly complementary to its target sequence to achieve sufficient specificity, i.e. to minimize non-specific binding of the oligonucleotide to non-target sequences under the particular binding conditions being used (e.g. in vivo physiological conditions or in vitro assay conditions). “Target RNA” refers to an RNA molecule of interest which is the target for hybridizing with/binding to an oligonucleotide of the invention to prevent or decrease for example the translation, reverse transcription and or replication of the RNA. In embodiments, such prevention and inhibition is via an induction of RNase H-mediated cleavage of the target RNA, and therefore in an embodiment, the invention provides a method of cleaving a target RNA, said method comprising contacting the RNA with an oligonucleotide of the invention. In embodiments, such cleavage may be further facilitated by additionally providing conditions conducive to RNase H activity, such as buffer means (e.g. to control pH and ionic strength), temperature control means, and any other components which may contribute to an induction in RNase H activity. In certain embodiments, RNase H activity is of an RNase H enzyme or of a multifunctional enzyme possessing RNase H activity. In certain embodiments, such RNase H activity includes, but is not limited to RNase H activity associated with the reverse transcriptases of human pathogenic viruses such as HIV (e.g. the retroviruses HIV-1 and HIV-2) and the hepadnavirus hepatitis B virus. In further embodiments, such RNase H activity includes, but is not limited to RNase H activity associated with an RNase H enzyme of prokaryotic or eukaryotic origin, in an embodiment, of mammalian origin, in an embodiment, of human origin. In further embodiments, such RNase H activity includes, but is not limited to RNase H activity associated with RNase H1 and RNase H2 of eukaryotic or prokaryotic origin.

In embodiments, the above-noted RNA includes messenger RNA, or viral genomic RNA, such that the oligonucleotide can specifically inhibit the biosynthesis of proteins encoded by the mRNA, or inhibit virus replication, respectively. Partial modifications to the oligonucleotide directed to the 5′ and/or 3′-terminus, or the phosphate backbone or sugar residues to enhance their antisense properties (e.g. nuclease resistance) are within the scope of the invention. As demonstrated in this invention (vide infra), these oligonucleotides meet one of the most important requirements for antisense therapeutics, i.e., they bind to target RNA forming an AON/RNA duplex that is recognized and degraded by human RNase H. Furthermore, as shown below, the efficiency by which the AON-X-AON chimera promotes. RNA cleavage is superior to that seen with AONs lacking the acyclic modification (X or Xₙ linker).

Therefore, applicants' results presented herein establish that [R¹—X]ₐ—R², AON-X-AON and AON-Xₙ-AON chimeras are excellent models of antisense agents, and should serve as therapeutics and/or valuable tools for studying and controlling gene expression in cells and organisms.

As such, in alternative embodiments, the invention provides antisense molecules that bind to, induce degradation of and/or inhibit the translation of a target RNA (e.g. mRNA). Examples of therapeutic antisense oligonucleotide applications, incorporated herein by reference, include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166,195 issued Nov. 24, 1992; U.S. Pat. No. 5,004,810, issued Apr. 2, 1991; U.S. Pat. No. 5,194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to the target RNA to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target RNA for antisense binding may include not only the information to encode a protein, but also associated ribonucleotides, which for example form the 5′-untranslated region, the 3′-untranslated region, the 5′ cap region and intron/exon junction ribonucleotides. A method of screening for antisense and ribozyme nucleic acids that may be used to provide such molecules as PLA₂ inhibitors of the invention is disclosed in U.S. Pat. No. 5,932,435.

Antisense molecules (oligonucleotides) of the invention may include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P(O)₂—O—CH₂). Oligonucleotides having morpholino backbone structures may also be used (U.S. Pat. No. 5,034,506). In alternative embodiments, antisense oligonucleotides may have a peptide nucleic acid (PNA, sometimes referred to as “protein” or “peptide” nucleic acid) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al., Science, 1991, 254, 1497 and U.S. Pat. No. 5,539,082). The phosphodiester bonds may be substituted with structures that are chiral and enantiomerically specific. Persons of ordinary skill in the art will be able to select other linkages for use in practice of the invention.

As noted above, oligonucleotides may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

Accordingly, in various embodiments, a modified oligonucleotide of the invention may be used therapeutically in formulations or medicaments to prevent or treat a disease characterized by the expression of a particular target RNA. In certain embodiments, such a target nucleic acid is contained in or derived from an infectious agent and/or is required for the function and/or viability and/or replication/propagation of the infectious agent. In certain embodiments, such an infectious agent is a virus, in certain embodiments, a retrovirus, in a further embodiment, HIV. In further embodiments the expression of such a target nucleic acid is associated with the diseases including but not limited to inflammatory diseases, diabetes, cardiovascular disease (e.g. restinosis), and cancer. The invention provides corresponding methods of medical treatment, in which a therapeutic dose of a modified oligonucleotide of the invention is administered in a pharmacologically acceptable formulation. In embodiments, an oligonucleotide may also be administered as a prodrug, whereby it is modified to a more active form at its site of action. Accordingly, the invention also provides therapeutic compositions comprising a modified oligonucleotide of the invention, and a pharmacologically acceptable excipient or carrier. The therapeutic composition may be soluble in an aqueous solution at a physiologically acceptable pH.

In an embodiment, such compositions include an oligonucleotide of the invention in a therapeutically or prophylactically effective amount sufficient to treat or prevent a disease characterized by the expression of a particular target nucleic acid, and a pharmaceutically acceptable carrier.

A “therapeutically effective amount” refers to an amount effective, at dosages and for periods of time necessary, to achieve the desired therapeutic result, such as a decrease in or a prevention of the expression of a particular target nucleic acid. A therapeutically effective amount of a modified nucleic acid of the invention may vary according to factors such as the disease state, age, sex, and weight of the individual, and the ability of the modified nucleic acid to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or treating a disease characterized by the expression of a particular target nucleic acid. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, an oligonucleotide of the invention can be administered in a time release formulation, for example in a composition which includes a slow release polymer. The modified oligonucleotide can be prepared with carriers that will protect the modified oligonucleotide against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound, such as an oligonucleotide of the invention, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, an oligonucleotide of the invention may be formulated with one or more additional compounds that enhance its solubility.

Since the oligonucleotides of the invention are capable of inducing the RNase H-mediated cleavage of a target RNA, thus decreasing the production of the protein encoded by the target RNA, the modified oligonucleotides of the invention may be used in any system where the selective inactivation or inhibition of a particular target RNA is desirable. As noted above, examples of such uses include antisense therapeutics, in which expression of the target RNA is associated with illness or disease.

A further example of such a use is the selective depletion of a particular target gene product in a system to study the phenotypic effect(s) of such depletion on the system. Observations made via such depletion studies may thus allow the determination of the function of the target gene product. In certain embodiments, such uses include “target validation”, in which the above-described strategy enables the confirmation as to whether a particular target nucleic acid is associated with a particular phenotype or activity, and thus allows “validation” of the target. The above noted system may be cell or cell-free; in vitro or in vivo; prokaryotic or eukaryotic.

The invention further provides commercial packages comprising an oligonucleotide of the invention. In certain embodiments, such commercial packages further comprise at least one of the following instructions for use of the oligonucleotide for (a) decreasing the expression of a target RNA sequence; (b) inducing the RNase H cleavage of a target RNA sequence; (c) preventing or treating a disease characterized by the expression of a particular RNA target; and (d) validating a particular gene target.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

Synthesis of Acyclic Precursors Suitable for their Incorporation into Oligonucleotides

Precursor to Acyclic Residue IIb

Dimethoxytrityl-O—CH₂CH₂CH₂CH₂—O—P(Ni—Pr₂)OCH₂CH₂CN (1) was purchased from ChemGenes Corp. (Ashland, Mass.), and was used as received for the synthesis of AON-X-AON chimeras (See Example 3).

Precursor to Acyclic Residues IIc and IId

The acyclic nucleoside residues (herein referred to as 2′,3′-seconucleotides) consist of a 1-[1,5-dihydroxy-4(S)-hydroxymethyl-3-oxapent-2(R)-yl]-uracil unit which has been appropriately protected and functionalized for oligonucleotide incorporation as described below.

### Step A. Synthesis of 5′-monomethoxytrityl-2′,3′-seco-β-D-ribouracil (2)

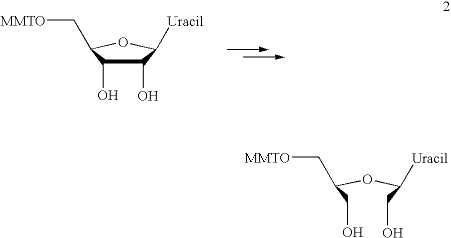

To a 0.1 M solution of 5′-monomethoxytrityluridine (5′-MMT-rU, 5.16 g, 10 mmol; prepared as described in T. Wu, K. K. Ogilvie, R. T. Pon. 1989. “Prevention of Chain Cleavage in the Chemical Synthesis of 2′-Silylated Oligoribonucleotides.” Nucleic Acids Res., 3501-17.) in dioxane was added a saturated solution of NaIO₄ in H₂O (2.26 g, 10.6 mmol, 1.06 eq) and the reaction allowed to proceed at r.t. for 2-3 h until complete conversion to the dialdehyde was observed by TLC visualization (R_{f} 0.52 in CH₂Cl₂:MeOH, 9:1). The reaction was diluted with dioxane (100 mL), filtered to remove NaIO₃ salts and followed by in situ reduction of the dialdehyde via treatment with NaBH₄ (0.378 g, 10 mmol, 1.0 eq) for 10-20 min. at r.t. The reaction mixture was quenched with acetone, neutralized with 20% acetic acid and concentrated to an oil under reduced pressure. The residue was then diluted with CH₂Cl₂ (200 mL) and washed with H₂O (2×75 mL). The aqueous layer was back-extracted and the combined organic layers were dried using anhydrous Na₂SO₄, filtered and evaporated to give the product as a pure white foam in 98% isolated yield (5.08 g; 9.8 mmol). R_{f} (CH₂Cl₂:MeOH, 9:1) 0.18; FAB-MS (NBA): 519.6; Calc: 518.57.

### Step B. Synthesis of 5′-O-MMT-2′-O-t-butyldimethysilyl-2′,3′-secouridine (3a) and 5′-O-MMT-3′-O-t-butyldimethysilyl-2′,3′-secouridine (3b)

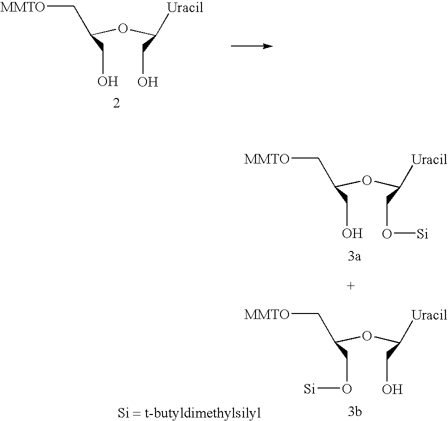

Monoprotection of either of the free hydroxyl functions of 2 was achieved nonselectively by adding t-butyldimethylsilyl chloride (0.81 g, 5.4 mmol, 1.1 eq) to a stirred 0.1 M solution of 2 (2.55 g, 4.9 mmol) in dry THF at 0° C. containing a suspension of AgNO₃ (0.92 g, 5.39 mmol, 1.1 eq). The reaction temperature was returned to r.t. after 20 min and maintained as such for 24 h. The workup was initiated by filtering the mixture directly into an aqueous solution of 5% NaHCO₃ (50 mL), followed by extraction of the aqueous layer twice with CH₂Cl₂. The combined organic layers were dried (anhydrous Na₂SO₄), filtered and evaporated under reduced pressure to give the crude product as a yellow oil. The residue was purified by flash silica gel column chromatography using a gradient of 0-25% acetone in CH₂Cl₂ to recover both monosilyl isomers as pure white foams. Isolated yields for 3a and 3b were 22% and 14%, respectively. R_{f} (CH₂Cl₂:Et₂O, 3:1) 3a, 0.18; 3b, 0.05. FAB-MS (NBA): 633.4; Calc: 632.83.

The regioisomers are distinguished on the basis of COSY-NMR cross-peak correlations that are used to demonstrate the connectivity of the protons in the acyclosugar. In both spectra, the H1′ protons are split by the nonequivalent H2′ and H2″ protons into a doublet of doublets which suggests a certain degree of structural rigidity around the C1′-C2′ bond. More significantly, a single well-resolved hydroxyl peak is observed for both 3a and 3b in DMSO-d₆ which negates rapid chemical exchange of these moieties. As a result, the effect of the protons at C2′ of 3b is transmitted to the 2′-hydroxyl proton which in turn appears as an overlapping doublet of doublets. In 3a, splitting of the hydroxyl peak is also observed, however it shows correlations with H4′ and H4″ and therefore rules out the presence of a silyl group at the 3′-position. Taken together, these data confirm the assignment of 3a and 3b as the 2′- and 3′-monosilylated isomers, respectively.

### Step C. (a) Synthesis of 5′-O-MMT-2′-O-t-butyldimethysilyl-2′,3′-secouridine-3′-O-[N,N-diisopropylamino-(2-cyanoethyl)]phosphoramidite (4a)

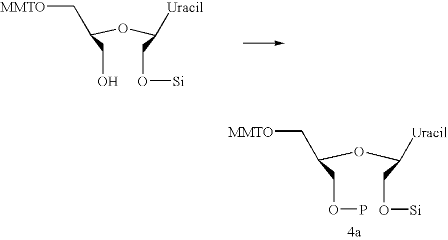 Si=t-butyldimethylsilyl; P=N,N-diisopropylamino-O-(2-cyanoethyl)phosphoramidite

To a nitrogen purged solution of 4-dimethylaminopyridine (DMAP; 12 mg, 0.10 mmol, 0.1 eq), N,N-diisopropylethylamine (DIPEA; 0.68 mL, 3.9 mmol, 4 eq) and 3a (620 mg, 0.98 mmol) in THF (0.2 M) at 0° C. was added N,N-diisopropylamino-β-cyanoethylphosphonamidic chloride (0.24 mL, 1.1 mmol, 1.1 eq) dropwise over 5 min. The immediate appearance of a white precipitate due to the rapid formation of diisopropylethylammonium hydrochloride signified sufficiently anhydrous conditions, and the reaction was allowed to warm to r.t. whereupon it was stirred for 2.5 h prior to the reaction workup. Briefly, the reaction mixture was diluted with EtOAc (50 mL, prewashed with 5% NaHCO₃) and washed with saturated brine (2×20 mL). The recovered organic layer was dried (anhydrous Na₂SO₄), filtered and the solvent removed via reduced pressure, yielding a crude yellow oil. Coevaporation of the crude product with Et₂O afforded a pale yellow foam. Purification of the product by flash silica gel column chromatography using a CH₂Cl₂:Hexanes:TEA gradient system (25:74:1 adjusted to 50:49:1) afforded a white foam in 97% isolated yield. R_{f} (EtOAc:Tol, 4:1) 0.86, 0.72. FAB-MS (NBA): 833.4; Calc: 833.05.

### (b) Synthesis of 5′-O-MMT-3′-O-t-butyldimethysilyl-2′,3′-secouridine-2′-O-[N,N-diisopropylamino-(2-cyanoethyl)]phosphoramidite (4b)

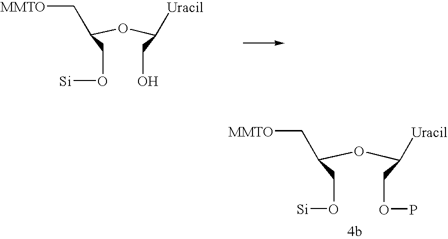 Si=t-butyldimethylsilyl; P=N,N-diisopropylamino-O-(2-cyanoethyl)phosphoramidite

All conditions used in the preparation of the 3′-phosphoramidite (4b) were identical to those performed on its regioisomeric counterpart, 4a (see step B). Flash column purification of this isomer afforded a white foam in 99% isolated yield. R_{f} (EtOAc:Tol, 4:1) 0.77, 0.65. FAB-MS (NBA): 833.3; Calc: 833.05.

### Example 2

Preparation of AONs Constructed from 2′-deoxy-2′-fluoro-β-D-arabinonucleotides (FANA) Flanking an Acyclic Butanediol or Secouridine Residues

1. Synthesis of FANA-X-FANA Chimeras, where X=Butanediol Linker=IIb (Y=Z=Oxygen; n=4)

The synthesis of PDE-FANA oligomers was conducted as previously described (Damha et al. J. Am. Chem. Soc. 1998, 120, 12976; Wilds, C. J. & Damha, M. J. Nucleic Acids Res. 2000, 28, 3625). Their structure was confirmed via Maldi-TOF mass spectrometry.

Synthesis of PDE-(FANA-But-FANA) chimeras were performed on a 1 micromole scale using an Expedite 8909 DNA-synthesizer. Long-chain alkylamine controlled-pore glass (LCAA-CPG) was used as the solid support. The synthesis cycle consisted of the following steps:
1)-Detritylation of nucleoside/tide bound to CPG (3% trichloroacetic acid/dichloroethane): 150 sec for MMT; 60 sec for DMT removal.
2) Coupling of 2′-F-arabinonucleoside or dimethoxytrityl-butanediol phosphoramidite monomers: 15 min. Concentration of monomers used were 50 mg/mL for araF-T, araF-C and 60 mg/mL for araF-A and butanediol monomers (acetonitrile as solvent).
3) Acetylation using the standard capping step: 20 sec. The capping solution consisted of 1:1 (v/v) of “capA” and “capB” reagents. CapA: acetic anhydride/collidine/THF (1:1:8 ml); cap B: N-Methylimidazole/THF (4:21 ml).
4) Extensive washing with acetonitrile (50 pulses).
5) Oxidation with a fresh solution of I₂:collidine:THF: 5 sec.
6) Washing with acetonitrile: 20 pulses.
7) Drying of the solid support by addition of the capping reagent (see step 3): 5 sec.
8) Washing with acetonitrile (20 pulses).

Following chain assembly, oligonucleotides were cleaved from the solid support and deprotected as previously described (Noronha, A. M. et al. Biochemistry 2000, 39, 7050). The crude oligomers were purified by anion-exchange HPLC followed by desalting (Sephadex G-25 or SepPak cartridges). Yields: 10-15 A₂₆₀ units

Conditions for HPLC Purification:
Column: Protein Pak DEAE-5PW (7.5 mm×7.5 cm, Waters),
Solvents: Buffer A: H₂O; Buffer B: 1M LiClO₄ (or NaClO₄),
Gradient: 0-20% B, linear over 60 min.

Loading was 0.5-1 A₂₆₀ units for analysis and 50-80 A₂₆₀ units for preparative separation. Flow rate was set at 1 mL/min, temperature was adjusted at 50° C. The detector was set at 260 nm for analytical and 290 nm for preparative chromatography. Under these conditions, the desired full-length oligomer eluted last.

The base sequence and hybridization properties of the oligonucleotides synthesized are given in Table 1.

2. Synthesis of FANA-X-FANA Chimeras, where X is Secouridine (SEC) Linker IIc.

Phosphodiester FANA-SEC-FANA and FANA-SEC-SEC-FANA chimeras were synthesized analogously to the butanediol chimeric constructs (vide supra) using a concentration of 50 mg/mL of 2′,3′-secouridine monomers for the coupling step. Yields of the oligonucleotides after their cleavage from the solid support, deprotection, purification (HPLC) and desalting (SepPak cartridges) as described above were 10 A₂₆₀ units. Their structure was confirmed via Maldi-TOF mass spectrometry.

The base sequence and hybridization properties of the oligonucleotides synthesized are given in Table 1.

3. Synthesis of DNA-X-DNA Chimeras, where X=Butanediol Linker=IIb (Y=Z=Oxygen, and n=4)

The synthesis and purification of phosphodiester DNA-But-DNA chimeras proceeded in the same manner as described above for phosphodiester FANA-But-FANA oligomers with few minor exceptions. The concentration of 2′-deoxyribonucleoside monomers and butanediol phosphoramidite used was 50 and 60 mg/mL, respectively in conjunction with a shorter coupling time (2 min) per addition of each type of monomer. Yields after purification (HPLC) and desalting (Sephadex G-25): 22 A₂₆₀ units. Their structure was confirmed by Maldi-TOF mass spectrometry.

The base sequence and hybridization properties of the oligonucleotides synthesized are given in Table 1.

4. Synthesis of Phosphorothioate FANA-X-FANA and Phosphorothioate DNA-X-DNA Chimeras, where X=Butanediol Linker=IIb (Y=Oxygen, Z=Sulfur, and n=4)

Synthesis of phosphorothioate FANA-But-FANA and phosphorothioate DNA-But-DNA oligomers was performed as described above for the phosphodiester (PDE) oligonucleotides. The main difference being the replacement of the iodine/water oxidation reagent with 0.1 M solution of 3-amino-1,2,4-dithiazoline-5-thione (ADTT) in pyridine/acetonitrile (1/1, v/v). Specifically, the phosphorothioate compounds were synthesized on a 1 micromol scale using an Expedite 8909 DNA-synthesizer. Long-chain alkylamine controlled-pore glass (LCAA-CPG) was used as the solid support. The synthesis cycle consisted of the following steps: (a) Detritylation of nucleoside/tide bound to CPG (3% trichloroacetic acid/dichloromethane): 150 sec.; (b) Coupling of 2′-F-arabinonucleoside or 2′-deoxyribonucleoside 3′-phosphoramidite monomers: 15 min or 1.5 min respectively. Concentration of monomers used were 50 mg/mL for araF-T, araF-C, DNA and butanediol linker monomers, and 60 mg/mL for araA and araF-G (acetonitrile as solvent); (c) Acetylation using the standard capping step: 20 sec. The capping solution consisted of 1:1 (v/v) of “capA” and “capB” reagents. CapA: acetic anhydride/collidine/THF (1:1:8 ml); cap B: N-Methylimidazole/THF (4:21 ml); (d) Extensive washing with acetonitrile (50 pulses); (e) Sulfurization with a solution of 0.1 M 3-amino-1,2,4-dithiazoline-5-thione (ADTT) in pyridine/acetonitrile (1/1, v/v), 10 min. (f) Washing with acetonitrile: 20 pulses; (g) Drying of the solid support by addition of the capping reagent (see step 3): 5 sec; (h) Washing with acetonitrile (20 pulses).

Following chain assembly, oligonucleotides were cleaved from the solid support and deprotected by treatment with conc. aqueous ammonia (r.t., 16 h). The crude oligomers were purified by either (a) preparative gel electrophoresis (24% acrylamide, 7M Urea) following by desalting (Sephadex G-25), or (b) anion-exchange HPLC following by desalting (SepPak cartridges). Yields: 30-70 A₂₆₀ units. Conditions for HPLC Purification: Column: Protein Pak DEAE-5PW (7.5 mm×7.5 cm, Waters), Solvents: Buffer A: H₂O; Buffer B: 1M NaClO₄, Gradient: 100% buffer A isocratic for 12 min, 100% A-15% B, linear (over 5 min), 15% B-55% B, linear (over 60 min); Flow rate was set at 1 ml/min, temperature was adjusted at 50° C. The detector was set at 260 nm for analytical and 290 nm for preparative chromatography. Under these conditions, the desired full-length oligomer eluted last. The structure of oligonucleotides was confirmed via Maldi-TOF mass spectrometry.

TABLE 1

Melting Temperatures (Tm) and RNase H Mediated
Hydrolysis Profiles for the AON/RNA Heteroduplexes^{a}

| | | | |
|---|---|---|---|
| | | | Relative rates |
| | SEQ ID | Tₘ | (kᵣₑₗ) of |
| Sequence type^{b,c} (5′ ± 3′) | NO: | (° C.) | enzyme cleavage^{d} |

| | | | | |
|---|---|---|---|---|
| | (i) DNA | | | |
| I | ttt ttt ttt ttt ttt ttt | 1 | 39 | 1 |
| II | ttt ttt ttt Btt ttt ttt | 2 | 33 | 2.7 |
| III | tta tat ttt ttc ttt ccc | 3 | 53 | 1 |
| IV | tta tat ttt Btc ttt ccc | 4 | 48 | 3.4 |
| V | tta tat ttt ctc ttt ccc | 5 | 40 | 0.7 |
| VI | tta tat ttt B ttc ttt ccc | 6 | 48 | 2.5 |
| | (ii) 2′F-ANA | | | |
| VII | TTT TTT TTT TTT TTT TTT | 7 | 53 | 1 |
| VIII | TTT TBT TTT TTT TTT TTT | 8 | 49 | 0.6 |
| IX | TTT TTT TTT BTT TTT TTT | 9 | 47 | 7.9 |
| X | TTT TTT TTT TTT BTT TTT | 10 | 49 | 5.1 |
| XI | TTT TTT TTT STT TTT TTT | 11 | 47 | 1.6 |
| XII | TTT TTT TTS STT TTT TTT | 12 | 42 | 2.8 |
| XIII | TTA TAT TTT TTC TTT CCC | 13 | 64 | 1 |
| XIV | TTA TAT TTT BTC TTT CCC | 14 | 55 | 3.5 |
| XV | TTA TAT TTT CTC TTT CCC | 15 | 55.5 | 0.9 |
| XVI | TTA TAT TTT tTC TTT CCC | 16 | 63 | 1.6 |
| XVII | TTA TAT TTT B TTC TTT CCC | 17 | 57 | 2.3 |
| | (iii) Ha-ras AON^{e} | | | |
| XVIII | att ccg tca tcg ctc ctc | 18 | 69.9 | 33.8 |
| XIX | att ccg tca Bcg ctc ctc | 19 | 58.2 | 31.6 |
| XX | att ccg tca ccg ctc ctc | 20 | 63.1 | 31.9 |
| XXI | ATT CCG TCA TCG CTC CTC | 21 | 82.0 | 1 |
| XXII | ATT CCG TCA BCG CTC CTC | 22 | 71.7 | 23.3 |
| | (iv) Phosphorothioate-AON sequences^{f} | | | |
| XXIII | tat tcc gtc atc gct cct ca | 23 | 64 | >>33 |
| XXIV | tat tcc gtc atc Bct cct ca | 24 | 50 | 32.7 |
| XXV | TAT TCC GTC ATC GCT CCT CA | 25 | 74 | 1 |
| XXVI | TAT TCC GTC ATC BCT CCT CA | 26 | 64 | 13 |

^{a}Aqueous solutions of 2.8 × 10⁻⁶ M of each oligonucleotide, 140 mM KCl, 1 M MgCl₂, 5 mM Na₂HPO₄ buffer (pH 7.2); uncertainty in Tₘ is ±0.5° C.
^{b}Target RNA sequences correspond to rA₁₈ (SEQ ID NO: 27), or 5′-r (GGGAAAGAAAAAAUAUAA)-3′(SEQ ID NO: 28).
^{c}Upper case letters, 2′F-ANA nucleotides; lower case letters, deoxynucleotides; c, arabinofluoro- or deoxycytidine mismatch residue; B, butanediol linker; S, 2′-secouridine insert.
^{d}Human enzyme; rates shown have been obtained at 22° C. and are normalized according to the parent strand of each series except within Ha-ras sequences in which data are normalized to all-FANA AON (entry XXI).
^{e,f}Target RNA (40 mer) sequence: 5′-r (CGCAGGCCCCUGAGGAGCGAUGACGGAAUAUAAGCUGGUG)-3′ (SEQ ID NO: 29); ^{e}underlined and ^{f}bold residues denote the region in the RNA to which the AON binds.

### Example 3

Expression and Purification of Human RNase HII

Expression of Human RNase HII

A hrnh gene fragment from pcDNA/GS/hrnh (Invitrogen) was obtained by PCR using the primers: AGC TAT CTC GAG ATG AGC TGG CTT CTG TTC CTG GCC (XhoI) (SEQ ID NO: 30), and GGC CGC AAG CTT TCA GTC TTC CGA TTG TTT AGC TCC (HindIII) (SEQ ID NO: 31). This fragment was cloned in the XhoI/HindIII sites of the bacterial expression vector pBAD/His (Invitrogen). Recombinant human RNase HII from pBAD/His/hrnh expression plasmid was purified as follows. To overcome codon bias in E. coli, we transformed the expression vector in E. coli BL21 codonplus (Stratagene) and cultured in LB broth containing 100 μg/ml ampicillin at 37° C. until the OD₆₀₀ reached 0.5-0.6. Then, the recombinant protein was induced with 0.002% arabinose for 4 h.

Purification of Human RNase HII

After induction, E. coli cells were harvested by centrifugation, washed in chilled wash buffer (100 mM phosphate, pH 8.0, 300 mM NaCl), resuspended in chilled lysis buffer (100 mM phosphate, pH 8.0, 10 Units/ml DNase, 2 mM phenylmethylsulfonyl fluoride, 300 mM NaCl, 200 μg/ml lysozyme), and lysed by 0.5% NP-40. The supernatant was applied to a Ni²⁺-nitrilotriacetate-agarose column after being centrifuged, and the protein was purified according to the manufacturer's directions (Qiagen). The eluate was treated with 2 mM EDTA to chelate any residual Ni²⁺ ions, dialyzed against 10 mM Tris-HCl, pH 8.0, and then concentrated by ultrafiltration. The protein was treated with enterokinase (1 unit/20 microgram of protein) in 50 mM Tris, pH 8.0, 1 mM CaCl₂, 0.1% Tween-20 overnight at 37° C. Then the digested sample was loaded on a Heparin-Sepharose column (Amersham-Pharmacia) pre-equilibrated with 20 mM phosphate buffer (pH 8.0). After elution with a gradient of 0.1-0.5 M NaCl, the desired peak was pooled, dialyzed, and concentrated.

### Example 4

Induction of Human Ribonuclease H(RNase HII) Activity by AON-X-AON Oligonucleotides

PDE-FANA Versus PDE-[FANA-X-FANA], where X=Butanediol Linker=IIb (Y=Z=Oxygen, and n=4)

A. Homopolymeric Sequences.

Defined-sequence oligonucleotides, 18-units in length, were used in these experiments:

| | |
|---|---|
| 5′-araF(TTT TTT TTT TTT TTT TTT)-3′ | (SEQ ID NO: 7) |
| T series “FANA” | |
| 5′-araF(TTT TTT TTT XTT TTT TTT)-3′ | (SEQ ID NO: 9) |
| T series ”FANA-But” | |

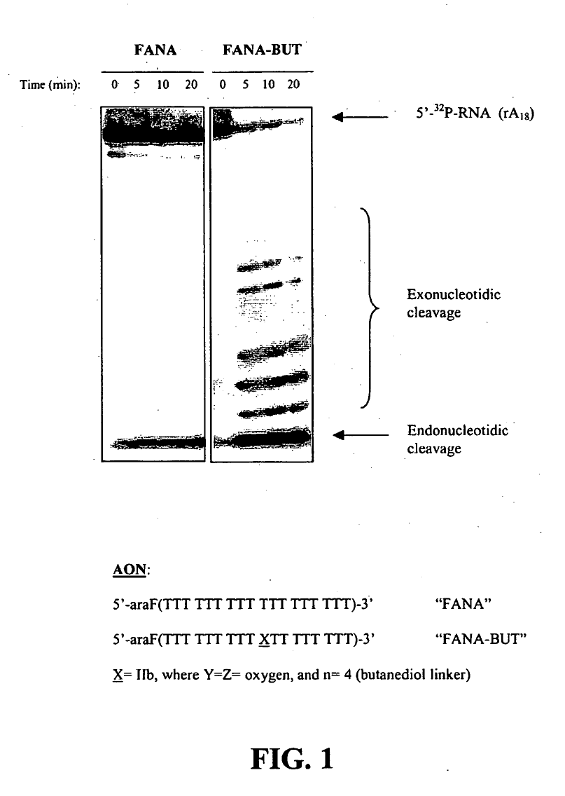

The residue X in the sequences above corresponds to acyclic residue IIb, where Y=Z=oxygen, and n=4 (or butanediol linker). The target RNA used was octadecariboadenylate (rA₁₈) complementary to the sequence of the above oligonucleotides. The ability of the above oligonucleotides to elicit RNase H degradation of target RNA was determined in assays (10 μL final volume) that comprised 1 pmol of 5′-[³²P] target RNA and 3 pmol of test oligonucleotide in 60 mM Tris-HCl containing 2 mM dithiothreitol, 60 mM KCl, and 2.5 mM MgCl₂ (pH 7.8, 22° C.). Prior to addition of the enzyme, the mixture was heated at 75° C. for 2 minutes, and then cooled slowly to room temperature to allow duplex formation. Reactions were started by the addition of human RNase HII at room temperature. Reactions were quenched by the addition of 10 μL of loading buffer (98% deionized formamide containing 10 mM EDTA and 1 mg/mL each of bromophenol blue and xylene cyanol), and heating at 100° C. for 5 minutes. The reaction products were resolved by electrophoresis using a 16% polyacrylamide sequencing gel containing 7 M urea, and visualized by autoradiography. The result of such an experiment is shown in FIG. 1.

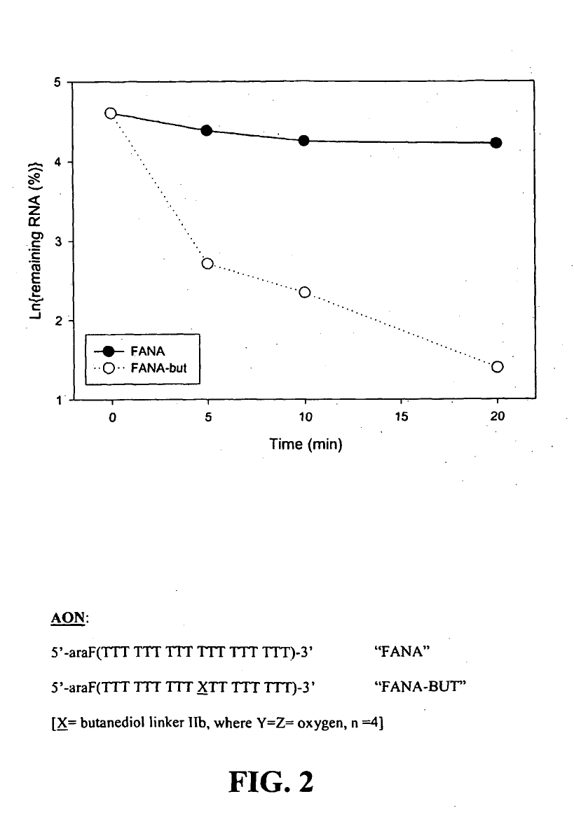

The results show that both “FANA” and “FANA-BUT” oligomers (T series) are able to form duplexes with target RNA that serve as substrates for the activity of human RNase HII, as indicated by the degradation products of the target RNA in FIG. 1. In the case of FANA this RNase H degradation was noted by the appearance of a fast moving band formed by the endonuclease activity of RNase HII. In the case of the “FANA-BUT” oligomer, degradation results from both the endo- and exonuclease activity of the enzyme, as evidenced by the appearance of numerous smaller sized RNA degradation products. Quantitation of rA₁₈ remaining as a function of time indicates that the rate of cleavage is 8 times faster with “FANA-But” than with “FANA” (TABLE 1 and FIG. 2).

The same trend was observed when mixed-based phosphodiester oligonucleotides were targeted against complementary RNA sequences (Examples 4B and 8). In Example 8, oligonucleotides containing the four naturally occurring heterocyclic bases (A, G, C and T) were designed to target 40- and 50-nt long RNA targets. In these cases, the rate enhancement of RNase H-mediated RNA cleavage observed was even more dramatic, reaching 23-fold in favor of the FANA-But-FANA over the FANA compounds.

B. Mixed Base Sequence

Defined-sequence oligonucleotides, 18-units in length, were used in these experiments:

(SEQ ID NO: 13)

5′-araF(TTA TAT TTT TTC TTT CCC)-3′
CAT “FANA”

(SEQ ID NO: 14)

5′-araF(TTA TAT TTT XTC TTT CCC)-3′
CAT “FANA-But”

(SEQ ID NO: 15)

5′-araF(TTA TAT TTT CTC TTT CCC)-3′
CAT “FANA-Mismatch”

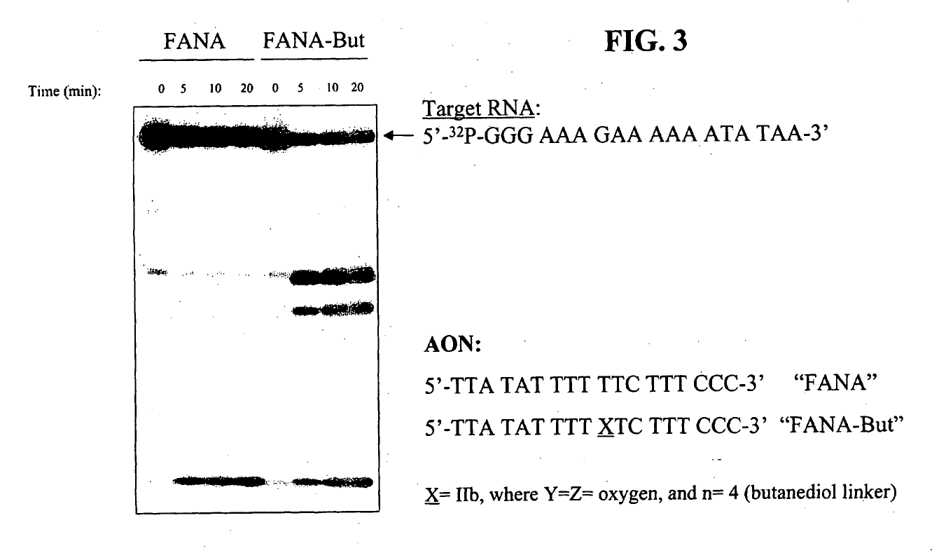

The residue X in the sequences above corresponds to acyclic residue IIb, where Y=Z=oxygen, and n=4 (or butanediol linker). The target RNA used was r(GGGAAAGAAAAAAUAUAA) (SEQ ID NO: 28), exactly complementary to the sequence of the first two oligonucleotides. The third oligonucleotide, CAT “FANA-Mismatch” contains an araF-C mismatch at position 10. This oligomer exhibits the same binding affinity as the “FANA-But” sequence, and was tested in order to assess the effect of a butanediol “linker” versus an araF-C mismatch “linker”. Therefore, the ability of “FANA”, “FANA-But”, and “FANA-Mismatch” (CAT series) to elicit RNase H degradation of target RNA was determined in assays (10 μL final volume) that comprised 1 pmol of 5′-[³²P] target RNA and 3 pmol of test oligonucleotide in 60 mM Tris-HCl containing 2 mM dithiothreitol, 60 mM KCl, and 2.5 mM MgCl₂ (pH 7.8, 22° C.). Assays were carried out as described above for the homopolymeric sequences (Example 4A). The result of such an experiment is shown in FIG. 3.

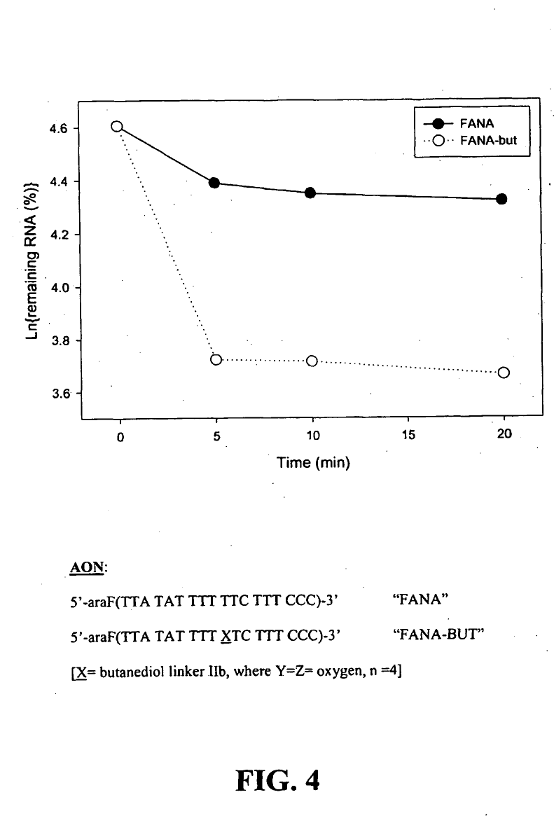

The results show that both “FANA” and “FANA-But” (CAT series) are able to form duplexes with target RNA that serve as substrates for the activity of human RNase HII, as indicated by the appearance of numerous smaller sized degradation products. Quantitation of RNA target remaining as a function of time indicate that the rate of cleavage is significantly faster (3.5 fold) with “FANA-But” than with “FANA” (FIG. 4).

The data also show that RNase H activity is diminished in the FANA-mismatch oligomer, where the more rigid araF-C “linker” replaces the more flexible butanediol linker (TABLE 1). Because the araF-C mismatch also induces an equivalent drop in duplex thermal stability relative to the butanediol insertion (ΔTm=−9° C., TABLE 1), it can be concluded that increased turnover (i.e., enhanced rate of dissociation from target RNA) is not the sole basis for preferential enzyme discrimination towards the more flexible But linker.

### Example 5

Human Ribonuclease H(RNAse HII) Activity as a Function of Position of Butanediol Linker IIb (Y=Z=Oxygen, and n=4)

The following oligonucleotides, 18-units in length, were used in these experiments:

(SEQ ID NO: 8)

5′-araF(TTT TXT TTT TTT TTT TTT)-3′
FANA “BUT 5”

(SEQ ID NO: 9)

5′-araF(TTT TTT TTT XTT TTT TTT)-3′
FANA “BUT 10”

(SEQ ID NO: 10)

5′-araF(TTT TTT TTT TTT XTT TTT)-3′
FANA “But 13”

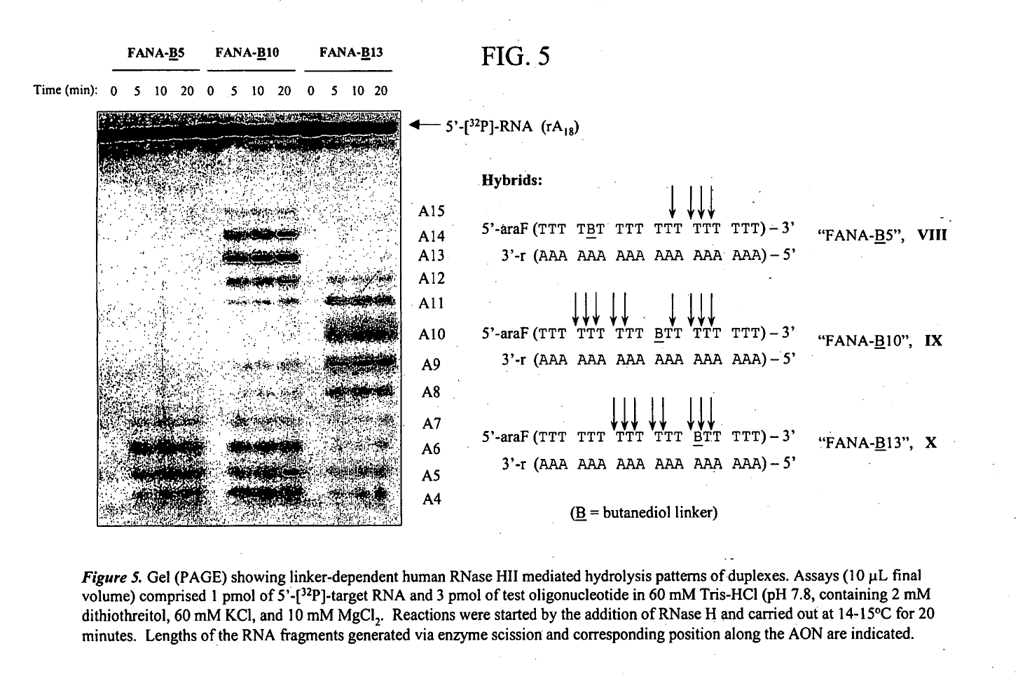

The X linkers are placed at various positions in order to determine whether optimal activity is dependent upon the location of the linker. X corresponds to acyclic residue Ib, where Y=Z=oxygen, and n=4 (butanediol linker). It was also desirable to determine the precise pattern and rate of cleavage that accompanies the movement of the linker along the FANA backbone. The exact location of primary cuts is difficult to measure under ambient temperature for the homopolymers, which are already known to be good substrates for the enzyme. As it was of interest to see where the first cuts were occurring, this information was instead extracted from assays conducted at the lower temperature, under which enzyme activity is retarded just enough to enable a qualitative comparison on the preferred cleavage modes toward each substrate (FIG. 5). At higher temperatures, the pattern becomes less interpretable as it results from the superimposition of multiple cleavages on a single target by the enzyme.

The target RNA used was rA₁₈, complementary to the above oligonucleotides. Assays (10 μL final volume) comprised 1 pmol of 5′-[³²P]-target RNA and 3 pmol of test oligonucleotide in 60 mM Tris-HCl (pH 7.8, containing 2 mM dithiothreitol, 60 mM KCl, and 10 mM MgCl₂. Reactions were started by the addition of RNase H and carried out at 14-15° C. for 20 minutes. The result of such an experiment is shown in FIG. 5.

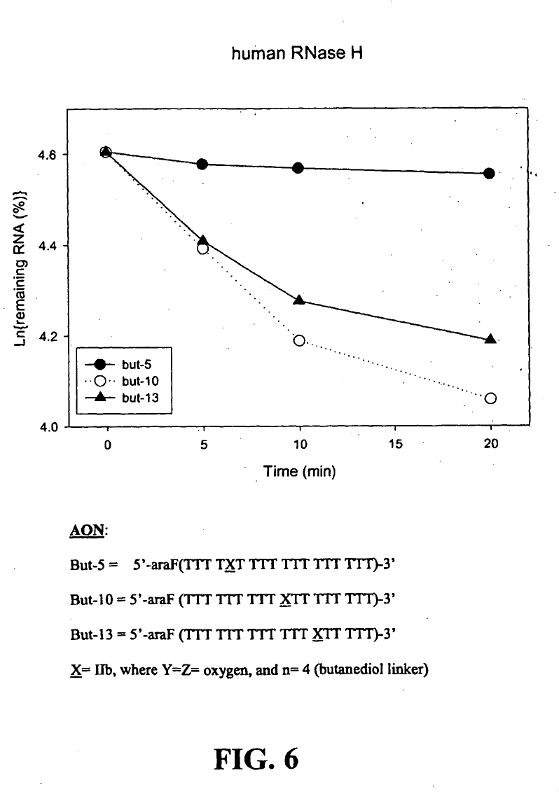

The relative rates follow the order: BUT-10>BUT-13>BUT-5 (TABLE 1 & FIG. 6). Furthermore, all of the linker-containing oligonucleotides induce additional primary cuts at the 3′-end of the RNA except for FANA-BUT5, which coincidentally, is the only oligonucleotide superceded in rate by the FANA oligomer, lacking the linker. As such, the FANA-BUT5 and FANA-BUT13 substrates show large differences in activation potency, in spite of the fact that their sequences are virtually identical and equally thermostable (TABLE 1), yet with opposite directionalities with respect to the butyl site in the oligonucleotide. Indeed, the different activities of these two oligomers suggest a minor—if not absent—role for the turnover effect. Alternatively, the diminished rate enhancement seen for FANA-BUT5 may reflect the remote positioning of RNase H along the substrate, which is known to bind near the 3′-end of the antisense oligonucleotide in the hybrid duplex and so may be unaffected by the linker insertion.

### Example 6

PDE-FANA Versus PDE-[FANA-X-FANA]-& PDE-[FANA-X-X-FANA] (X=Seconucleotide IIc)

Defined-sequence oligonucleotides, 18-units in length, were used in these experiments:

(SEQ ID NO: 7)

5′-araF(TTT TTT TTT TTT TTT TTT)-3′
“FANA”

(SEQ ID NO: 11)

5′-araF(TTT TTT TTT XTT TTT TTT)-3′
“SECx1”

(SEQ ID NO: 12)

5′-araF(TTT TTT TTX XTT TTT TTT)-3′
“SECx2”

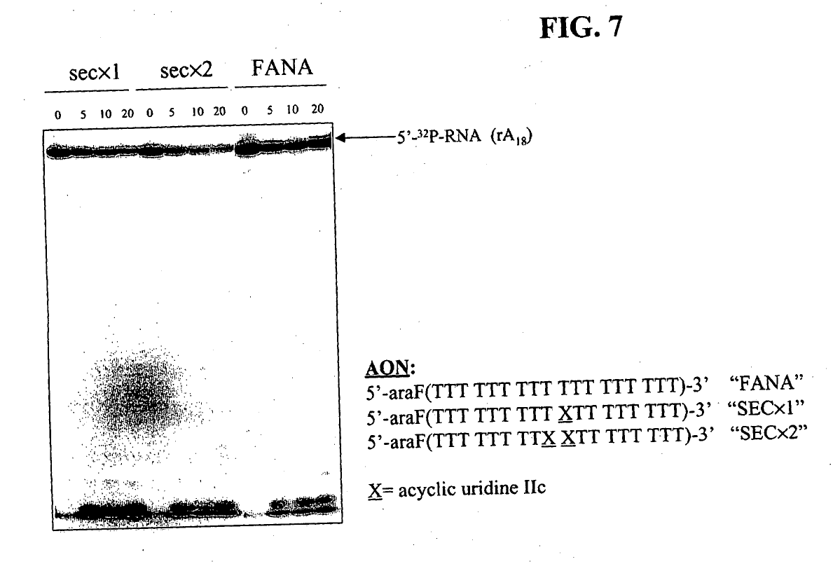

The residue X in the sequences above corresponds to acyclic residue IIc (secouridine). The target RNA used was octadecariboadenylate (rA₁₈) complementary to the sequence of the above oligonucleotides. The ability of the above oligonucleotides to elicit RNase H degradation of target RNA was determined in assays (10 μL final volume) that comprised 1 pmol of 5′-[³²P] target RNA and 0.3 pmol of test oligonucleotide in 60 mM Tris-HCl containing 2 mM dithiothreitol, 60 mM KCl, and 2.5 mM MgCl₂ (pH 7.8, 15° C.). Prior to addition of the enzyme, the mixture was heated at 75° C. for 2 minutes, and then cooled slowly to room temperature to allow duplex formation. Reactions were started by the addition of human RNase HII at room temperature. Reactions were quenched by the addition of 10 μL of loading buffer (98% deionized formamide containing 10 mM EDTA and 1 mg/mL each of bromophenol blue and xylene cyanol), and heating at 100° C. for 5 minutes. The reaction products were resolved by electrophoresis using a 16% polyacrylamide sequencing gel containing 7 M urea, and visualized by autoradiography. The result of such an experiment is shown in FIG. 7.

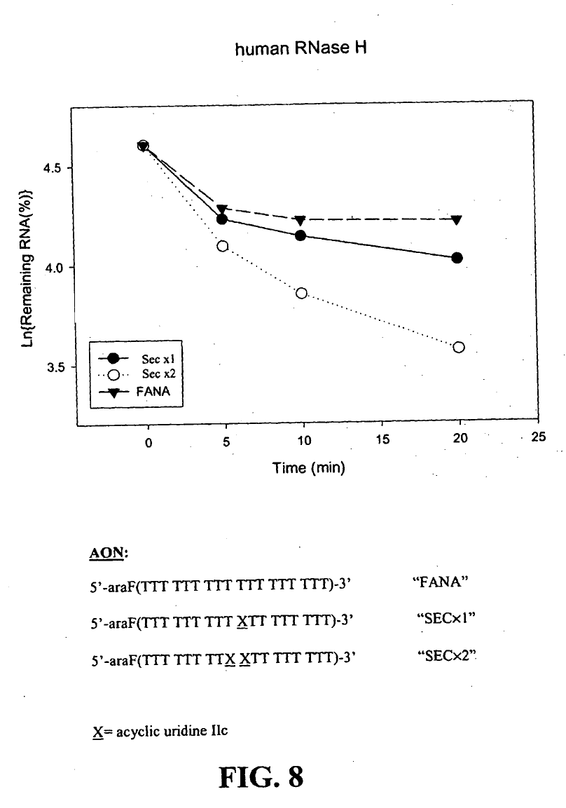

The results show that all “FANA”, “SEC×1” and “SEC×2” are able to form duplexes with target RNA that serve as substrates for the activity of human RNase HII, as indicated by the disappearance (degradation) of the band corresponding to the full length target RNA (FIG. 7). Quantitation of rA₁₈ remaining as a function of time indicates that the rate of RNA cleavage is greater when the hybridized AON is “SEC×2” (FIG. 8). The order observed is “FANA-SEC×2”>“FANA-SEC×1”>“FANA”, demonstrating that an unprecedented enhancement in targeted RNA cleavage is imparted to the parent FANA strand by the seconucleotide linkers (IIc). As for the butanediol insertions (Example 5), the same trend is observed—i.e. reduced thermal stability relative to the all-FANA counterpart, yet enhanced RNase H activity. Thus, the drop in melting temperature caused by linker insertions is outweighed by the observed rate enhancement of the target RNA relative to the all-FANA constructs.

### Example 7

PDE-DNA Versus PDE-[DNA-X-DNA] (X=Butanediol Linker=IIb, Y=Z=Oxygen, and n=4)

A. Homopolymeric Sequences.

Defined-sequence oligonucleotides, 18-units in length, were used in these experiments:

| | |
|---|---|
| 5′-d(TTT TTT TTT TTT TTT TTT)-3′ | (SEQ ID NO: 1) |
| “DNA” | |
| 5′-d(TTT TTT TTT XTT TTT TTT)-3′ | (SEQ ID NO: 2) |
| “DNA-But” | |

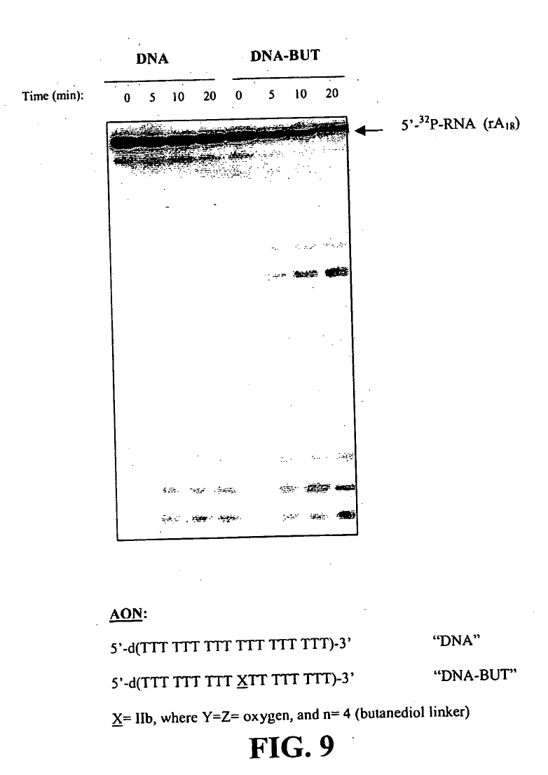

The residue X in the sequences above corresponds to acyclic residue IIb, where Y=Z=oxygen, n=4 (or butanediol linker). The target RNA used was octadecariboadenylate (rA₁₈) complementary to the sequence of the above oligonucleotides. The ability of the above oligonucleotides to elicit RNase H degradation of target RNA was determined in assays (10 μL final volume) that comprised 1 pmol of 5′-[³²P]-target RNA and 3 pmol of test oligonucleotide in 60 mM Tris-HCl containing 2 mM dithiothreitol, 60 mM KCl, and 2.5 mM MgCl₂ (pH 7.8, 15° C.). Assays were carried out as described above for Example 4A. The result of such an experiment is shown in FIG. 9.

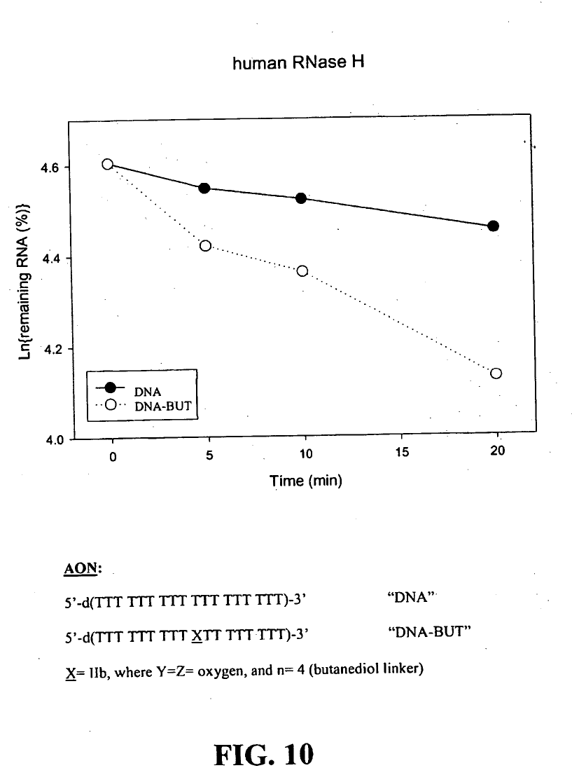

The results show that both “DNA” and “DNA-BUT” oligomers are able to form duplexes with target RNA that serve as substrates for the activity of human RNase HII, as indicated by the degradation products of the target RNA in FIG. 9. Quantitation of rA₁₈ remaining as a function of time indicates that the rate of cleavage is significantly faster (ca. 3-fold) with “DNA-BUT” than with “DNA” (FIG. 10).

B. Mixed Base Sequence

Defined-sequence oligonucleotides, 18-units in length, were used in these experiments:

| | |
|---|---|
| 5′-d(TTA TAT TTT TTC TTT CCC)-3′ | (SEQ ID NO: 3) |
| CAT “DNA” | |
| 5′-d(TTA TAT TTT XTC TTT CCC)-3′ | (SEQ ID NO: 4) |
| CAT “DNA-But” | |
| 5′-d(TTA TAT TTT CTC TTT CCC)-3′ | (SEQ ID NO: 5) |
| CAT “DNA-Mismatch” | |

The residue X in the sequences above corresponds to acyclic residue IIb, where Y=Z=oxygen, and n=4 (or butanediol linker). The target RNA used was r(GGGAAAGAAAAAAUAUAA) (SEQ ID NO: 28), exactly complementary to the sequence of the first two DNA oligonucleotides. The third oligonucleotide, CAT “DNA-Mismatch” contains a dC mismatch at position 10. The ability of “DNA”, “DNA-But”, and “DNA-Mismatch” (CAT series) to elicit RNase H degradation of target RNA was determined in assays (10 μL final volume) that comprised 1 pmol of 5′-[³²P] target RNA and 3 pmol of test oligonucleotide in 60 mM Tris-HCl containing 2 mM dithiothreitol, 60 mM KCl, and 2.5 mM MgCl₂ (pH 7.8, 15° C.). Assays were carried out as described above for Example 4A.

The kinetic data given in TABLE 1 show that all DNA oligomers are able to form duplexes with target RNA that serve as substrates for the activity of human RNase HII. Quantitation of RNA target remaining as a function of time indicates that the rate of cleavage is significantly faster with “DNA-BUT” than with “DNA” or “DNA-Mismatch” (3 and 4-fold, respectively; TABLE 1).

### Example 8

Targeting Higher Molecular Weight RNA. Comparison Between Phosphodiester FANA, FANA-X-FANA, DNA, Mismatched DNA, and DNA-X-DNA (X=Butanediol Linker=IIb, Y=Z=Oxygen, and n=4).

The following phosphodiester oligonucleotides, 18-units in length, were used in these experiments:

| | |
|---|---|
| 5′- d(ATT CCG TCA CTC CTC)-3′ | (SEQ ID NO: 18) Ha-RAS |
| “PDE-DNA” | |
| 5′- d(ATT CCG TCA XCG CTC CTC)-3′ | (SEQ ID NO: 19) Ha-RAS |
| “PDE-DNA-But” | |
| 5′- d(ATT CCG TCA CCG CTC CTC)-3′ | (SEQ ID NO: 20) Ha-RAS |
| “PDE-DNA-Mismatch” | |
| 5′-araF(ATT CCG TCA TCG CTC CTC)-3′ | (SEQ ID NO: 21) Ha-RAS |
| “PDE-FANA” | |
| 5′-araF(ATT CCG TCA XCG CTC CTC)-3′ | (SEQ ID NO: 22) Ha-RAS |
| “PDE-DNA-But” | |

The residue X in the sequences above corresponds to acyclic residue IIb, where Y=Z=oxygen, n=4 (or butanediol linker). The target RNA used was a polyribonucleotide 40 nucleotide units in length. Their base sequences are derived from the naturally occurring Ha-Ras mRNA sequence (derived from the c-ras protooncogene). The ability of each of the above oligonucleotides to elicit RNase H degradation of target RNA was determined in assays (10 μL final volume) that comprised 1 pmol of 5′-[³²P] _target RNA and 3 pmol of test oligonucleotide in 60 mM Tris-HCl containing 2 mm dithiothreitol, 60 mM KCl, and 2.5 mM MgCl₂ (pH 7.8). Reactions were started by the addition of RNase H and carried out at 37° C. Timed aliquots were taken at various time intervals from each set of incubation.

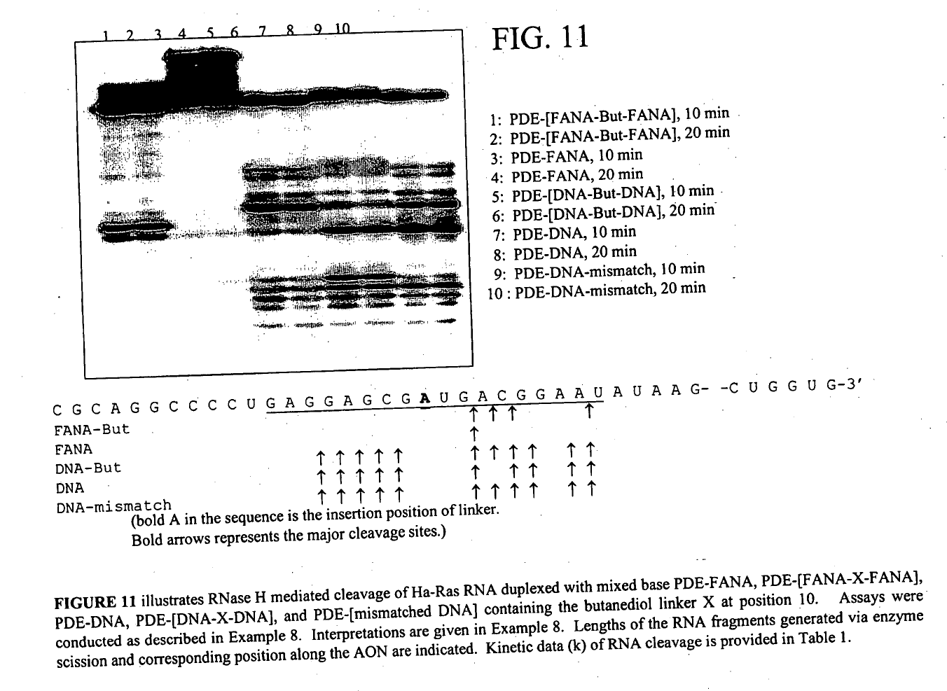

For this particular sequence, an increase in target degradation is not apparent upon interchanging a deoxynucleotide residue in DNA for a butanediol linker (oligomers XVIII and XIX, TABLE 1, and FIG. 11). However, this is not the case for the FANA constructs. As demonstrated in all of the previous Examples, substitution of the arabinofluoronucleoside residue in phosphodiester FANA with a more flexible butanediol linker elevates the activity of RNase HII. In fact, such a substitution closes the efficiency gap between FANA (kᵣₑₗ 23.3) and DNA-derived (kᵣₑₗ 33.8) antisense compounds considerably (TABLE 1).

### Example 9

Targeting Higher Molecular Weight RNA. Comparison Between PS-FANA, PS-[FANA-X-FANA], PS-DNA, and PS-[DNA-X-DNA] (X=Butanediol Linker=IIb, Y=Oxygen, Z=Sulfur, and n=4).

The following phosphorothioate oligonucleotides, 18-units in length, were used in these experiments:

| | |
|---|---|
| 5′ d(ATT CCG TCA TCG CTC CTC)-3′ | (SEQ ID NO: 23) Ha-RAS |
| “PS-DNA” | |
| 5′ d(ATT CCG TCA XCG CTC CTC)-3′ | (SEQ ID NO: 24) Ha-RAS |
| “PS-DNA-But” | |
| 5′-araF(ATT CCG TCA TCG CTC CTC)-3′ | (SEQ ID NO: 25) Ha-RAS |
| “PS-FANA” | |
| 5′-araF(ATT CCG TCA XCG CTC CTC)-3′ | (SEQ ID NO: 26) Ha-RAS |
| “PS-DNA-But” | |

The residue X in the sequences above corresponds to acyclic residue IIb, where Y=oxygen, Z=sulfur, n=4 (butanediol linker). The target RNA used was a polyribonucleotide 40 nucleotide units in length. Their base sequences are derived from the naturally occurring Ha-Ras mRNA sequence (derived from the c-ras protooncogene) The ability of each of the above oligonucleotides to elicit RNase H degradation of target RNA was determined in assays (10 μL final volume) that comprised 1 pmol of 5′-[³²P]-target RNA and 3 pmol of test oligonucleotide in 60 mM Tris-HCl containing 2 mM dithiothreitol, 60 mM KCl, and 2.5 mM MgCl₂ (pH 7.8). Reactions were started by the addition of RNase H and carried out at 37° C. Timed aliquots were taken at various time intervals from each set of incubation.

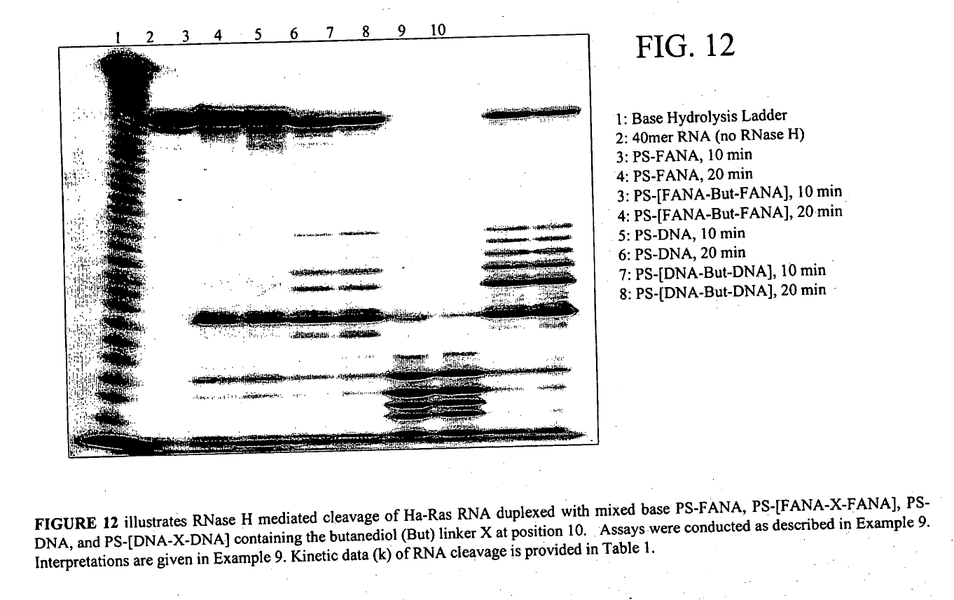

For this particular sequence, a decrease in target degradation is apparent upon interchanging a deoxynucleotide residue in DNA for a butanediol linker (TABLE 1, and FIG. 12). This is in contrast to what is observed for the FANA based constructs. In this case, substitution of the arabinofluoronucleoside residue in PS-FANA with a more flexible butanediol linker elevates the activity of RNase HII (TABLE 1).

### Example 10

Oligonucleotide Constructs Containing ‘Looping Out’ Acyclic Linkers

(SEQ ID NO: 13)

5′araF(TTA TAT TTT TTC TTT CCC)-3′
CAT “FANA”

(SEQ ID NO: 14)

5′-araF(TTA TAT TTT X TC TTT CCC)-3′
CAT “FANA-But”

(SEQ ID NO: 17)

5′-araF(TTA TAT TTT X TTC TTT CCC)-3′
CAT “FANA-But-loop”

The residue X in the sequences above corresponds to acyclic residue IIb, where Y=Z=oxygen, n=4 (or butanediol linker). The target RNA used was r(GGGAAAGAAAAAAUAUAA) (SEQ ID NO: 28), exactly complementary to each of the above-sequences. The ability of phosphodiester linked “FANA”, “FANA-But”, and “FANA-But-loop” (CAT series) to elicit RNase H degradation of target RNA was determined in assays (10 μL final volume) that comprised 1 pmol of 5′-[³²P] target RNA and 3 pmol of test oligonucleotide in 60 mM Tris-HCl containing 2 mM dithiothreitol, 60 mM KCl, and 2.5 mM MgCl₂ (pH 7.8, 15° C.). Assays were carried out as described above for Example 4A.

The “FANA-But-loop” sequence contains unifying elements of both the “FANA” and “FANA-But” oligonucleotides. These consist of a localized flexible site in the center of the sequence (similar to “FANA-But”) as well as the ability of this oligonucleotide to fully hybridize with the target RNA (similar to “FANA”). As a result, the looping linker likely extends away from the duplex core to maximize the number of residues that form base pairs between this particular sequence and the RNA. Forcing the linker out of the helix in this way may disrupt some of the interactions between RNase H and the two strands by reducing the number of stable contacts between the enzyme and the duplex minor groove. Surprisingly, this sequence still considerably enhances RNA degradation (compare oligomers XIII and XVII, TABLE 1), which suggests that flexibility in the antisense strand is important for effective RNase H induction, irrespective of whether the flexible linker resides directly within or away from the helix axis.

Throughout this application, various references describe the state of the art to which this invention pertains. The disclosures of these references are hereby incorporated by reference into the present disclosure.

### ABBREVIATIONS

ANA, arabinonucleic acid derivative (with a variable 2′-substituent)
AON, antisense oligonucleotide
BUT, 1,4-butanediol unit
DMSO, dimethylsulfoxide
DNA, deoxyribonucleic acid
EC₅₀, effective concentration
EDTA, ethylenediaminetetraacetate
Et₂O, diethyl ether
EtOAc, ethyl acetate
FAB-MS, fast-atom bombardment mass spectrometry
FANA, 2′-deoxy-2′-fluoroarabinonucleic acid
HPLC, high performance liquid chromatography
LCAA-CPG, long-chain alkylamine controlled pore glass
MBO, mixed-backbone oligonucleotide
MeOH, methanol
NBA, p-nitrobenzyl alcohol
O-PNA monomer, NH₂—CH(CH₂—CH₂-Base)-CH₂—O—CH₂—CO₂H
PDE-DNA, phosphodiester linked DNA
PNA, peptide nucleic acid
PNA monomer, N-(2-aminoethyl)glycine unit in which an heterocyclic base is attached via a methylene carbonyl linker.
PS-DNA, phosphorothioate linked DNA
R_{f}, retention factor
RNA, ribonucleic acid
RNase H, ribonuclease H
SEC, seconucleotide unit
TEA, triethylamine
THF, tetrahydrofuran
Tₘ, melting temperature
TLC, thin-layer chromatography
Tol, toluene

1. An oligonucleotide having the structure:
[R¹—X]ₐ—R² Ia
wherein a is an integer greater than or equal to 1;
wherein either R¹, R² each independently comprise at least one nucleotide;
wherein X is an acyclic linker; and
wherein said oligonucleotide comprises at least one modified deoxyribonucleotide.

2. The oligonucleotide of claim 1 wherein the modified deoxyribonucleotide is selected from the group consisting of ANA, PS-ANA, PS-DNA, RNA-DNA and DNA-RNA chimeras, PS-[RNA-DNA] and PS-[DNA-RNA] chimeras, PS-[ANA-DNA] and PS-[DNA-ANA] chimeras, RNA, PS-RNA, PDE- or PS-RNA analogues, locked nucleic acids (LNA), phosphorodiamidate morpholino nucleic acids, N3′-P5′ phosphoramidate DNA, cyclohexene nucleic acid, alpha-L-LNA, boranophosphate DNA, methylphosphonate DNA, and combinations thereof.

3. The oligonucleotide of claim 2 wherein the ANA is FANA.

4. The oligonucleotide of claim 3 wherein the FANA is selected from the group consisting of PDE-FANA and PS-FANA.

5. The oligonucleotide of claim 2, wherein the PDE- or PS-RNA analogues are selected from the group consisting of 2′-modified RNA wherein the 2′-substituent is selected from the group consisting of alkyl, alkoxy, alkylalkoxy, F and combinations thereof.

6. The oligonucleotide of claim 1, wherein the acyclic linker is selected from the group consisting of an acyclic nucleoside and a non-nucleotidic linker.

7. The oligonucleotide of claim 6, wherein the acyclic nucleoside is selected from the group consisting of purine and pyrimidine seconucleosides.

8. The oligonucleotide of claim 7 wherein the purine seconucleoside is selected from the group consisting of secoadenosine and secoguanosine.

9. The oligonucleotide of claim 7 wherein the pyrimidine seconucleoside is selected from the group consisting of secothymidine, secocytidine and secouridine.

10. The oligonucleotide of claim 1, wherein the non-nucleotidic linker comprises a linker selected from the group consisting of an amino acid and an amino acid derivative.

11. The oligonucleotide of claim 10, wherein the amino acid derivative is selected from the group consisting of (a) an N-(2-aminoethyl)glycine unit in which an heterocyclic base is attached via a methylene carbonyl linker (PNA monomer); and (b) an O-PNA unit.

12. The oligonucleotide of claim 1, wherein said oligonucleotide has the structure: 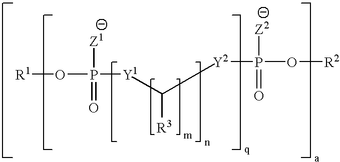
wherein each of m, n, q and a are independently integers greater than or equal to 1;
wherein each of R¹ and R² are independently at least one nucleotide;
wherein each of Z¹ and Z² are independently selected from the group consisting of an oxygen atom, a sulfur atom, an amino group and an alkylamino group;
wherein each of Y¹ and Y² are independently selected from the group consisting of oxygen, sulfur and NH; and
wherein R³ is selected from the group consisting of H, alkyl, hydroxyalkyl, alkoxy, a purine, a pyrimidine and combinations thereof.

13. The oligonucleotide of claim 12, wherein said purine is selected from the group consisting of adenine, guanine, and derivatives thereof.

14. The oligonucleotide of claim 12, wherein said pyrimidine is selected from the group consisting of thymine, cytosine, 5-methylcytosine, uracil, and derivatives thereof.

15. The oligonucleotide of claim 1, wherein each of R¹ and R² independently comprise at least two nucleotides having an internucleotide linkage, wherein said internucleotide linkage is selected from the group consisting of phosphodiester, phosphotriester, phosphorothioate, methylphosphonate, phosphoramidate (5′N-3′P and 5′P-3′N), and combinations thereof.

16. The oligonucleotide of claim 12, wherein each of R¹ and R² independently comprise ANA.

17. The oligonucleotide of claim 16, wherein said ANA comprises a 2′-substituent selected from the group consisting of fluorine, hydroxyl, amino, azido, alkyl, alkenyl, alkynyl, and alkoxy groups.

18. The oligonucleotide of claim 17, wherein said 2′-substituent is fluorine and said ANA is FANA.

19. The oligonucleotide of claim 17, wherein said alkyl group is selected from the group consisting of methyl, ethyl, propyl and butyl groups.

20. The oligonucleotide of claim 17, wherein said alkoxy group is selected from the group consisting of methoxy, ethoxy, propoxy, and methoxyethoxy groups.

21. The oligonucleotide of claim 12, wherein said oligonucleotide is selected from the group consisting of: 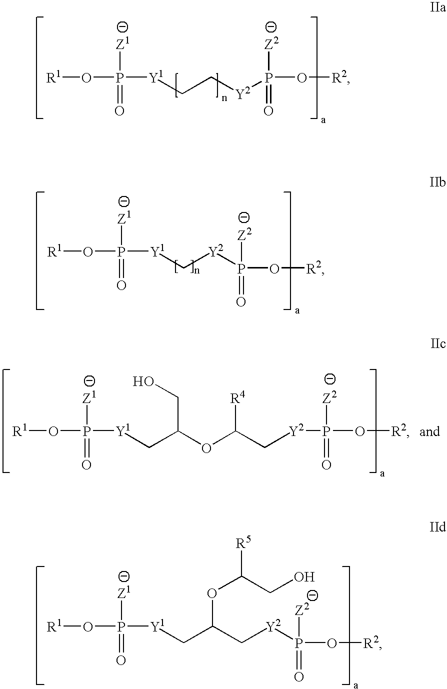
wherein n, a, R¹, R², Z¹, Z², Y¹ and Y² are as defined in claim 12; and
wherein each of R⁴ and R⁵ are independently selected from the group consisting of a purine and a pyrimidine.

22. The oligonucleotide of claim 21, wherein said purine is selected from the group consisting of adenine, guanine and derivatives thereof.

23. The oligonucleotide of claim 21, wherein said pyrimidine is selected from the group consisting of thymine, cytosine, uracil, and derivatives thereof.

24. The oligonucleotide of claim 1; wherein R¹ and R² are FANA; and wherein a=1.

25. The oligonucleotide of claim 1; wherein R¹ and R² are PS-DNA; and wherein a=1.

26. The oligonucleotide of claim 1; wherein R¹ is [FANA-DNA]; wherein R² is [DNA-FANA]; and wherein a=1.

27. The oligonucleotide of claim 1; wherein R¹ is [FANA-DNA]; wherein R² is FANA; and wherein a=1.

28. The oligonucleotide of claim 1; wherein R¹ is FANA; wherein R² is [DNA-FANA]; and wherein a=1.

29. The oligonucleotide of claim 1; wherein R¹ is [RNA-DNA]; wherein R² is [DNA-RNA]; and wherein a=1.

30. The oligonucleotide of claim 1; wherein R¹ is [RNA-DNA]; wherein R² is RNA; and wherein a=1.

31. The oligonucleotide of claim 1; wherein R¹ is RNA; wherein R² is [DNA-RNA]; and wherein a=1.

32. The oligonucleotide of claim 1; wherein R¹ is S-[(2′O-alkyl)RNA-DNA]; wherein R² is S-[DNA-(2′O-alkyl)RNA]; and wherein a=1.

33. The oligonucleotide of claim 1; wherein R¹ is S-[(2′O-alkyl)RNA-DNA]; wherein R² is S-[(2′O-alkyl)RNA]; and wherein a=1.

34. The oligonucleotide of claim 1; wherein R¹ is S-[(2′O-alkyl)RNA]; wherein R² is S-[DNA-(2′O-alkyl)RNA]; and wherein a=1.

35. The oligonucleotide of claim 1; wherein R¹ is S-[(2′O-alkoxyalkyl)RNA-DNA]; wherein R² is S-[DNA-(2′O-alkoxyalkyl)RNA]; and wherein a=1.

36. The oligonucleotide of claim 1; wherein R¹ is S-[(2′O-alkoxyalkyl)RNA-DNA]; wherein R² is S-[(2′O-alkoxyalkyl)RNA]; and wherein a=1.

37. The oligonucleotide of claim 1; wherein R¹ is S-[(2′O-alkoxyalkyl)RNA]; wherein R² is S-[DNA-(2′O-alkoxyalkyl)RNA]; and wherein a=1.

38. The oligonucleotide of claim 20; wherein R¹ is FANA; wherein R² is PS-FANA; wherein a=1; and wherein said oligonucleotide has structure IIb in which Y¹, Y², Z¹ and Z² are oxygen and n=4.

39. The oligonucleotide of claim 20; wherein R¹ is PS-FANA; wherein R² is FANA; wherein a=1; and wherein said oligonucleotide has structure IIb in which Y¹, Y², Z² are oxygen, and Z¹ are sulfur and n=4.

40. The oligonucleotide of claim 20; wherein R¹ is PS-DNA; wherein R² is DNA; wherein a=1; and wherein said oligonucleotide has structure IIb in which Y¹, Y², Z² are oxygen, Z² is sulfur and n=4.

41. The oligonucleotide of claim 20; wherein R¹ is DNA; wherein R² is PS-DNA; wherein a=1; and wherein said oligonucleotide has structure IIb in which Y¹, Y², Z¹ are oxygen, Z² is sulfur and n=4.

42. The oligonucleotide of claim 20; wherein R¹ is PS-FANA;
wherein R² is FANA; wherein a=1; and wherein said oligonucleotide has structure IIc.

43. The oligonucleotide of claim 20; wherein R¹ is FANA; wherein R² is PS-FANA; wherein a=1; and wherein said oligonucleotide has structure IIc.

44. The oligonucleotide of claim 20; wherein R¹ is PS-DNA; wherein R² is DNA; wherein a=1; and wherein said oligonucleotide has structure IIc.

45. The oligonucleotide of claim 20; wherein R¹ is DNA; wherein R² is PS-DNA; wherein a=1; and wherein said oligonucleotide has structure IIc.

46. The oligonucleotide of claim 1, wherein a=2 and each of R¹ and R² independently consist of at least 3 nucleotides.

47. The oligonucleotide of claim 46, wherein each of R¹ and R² independently consist of 3-8 nucleotides.

48. The oligonucleotide of claim 1, wherein a=3 and each of R¹ and R² independently consist of at least 2 nucleotides.

49. The oligonucleotide of claim 48, wherein each of R¹ and R² independently consist of 2-6 nucleotides.

50. The oligonucleotide of claim 1, wherein said oligonucleotide is antisense to a target RNA.

51. A method of preventing or decreasing translation, reverse transcription and/or replication of a target RNA in a system, said method comprising contacting said target RNA with the oligonucleotide of claim 50.

52. A method of preventing- or decreasing translation, reverse transcription and/or replication of a target RNA in a system, said method comprising:
a) contacting said target RNA with the oligonucleotide of claim 50; and
b) allowing RNase cleavage of said target RNA.

53. Use of the oligonucleotide according to claim 50 for preventing or decreasing translation, reverse transcription and/or replication of a target RNA in a system.

54. A commercial package comprising the oligonucleotide according to claim 50 together with instructions for its use for preventing or decreasing translation, reverse transcription and/or replication of a target RNA in a system.
